SEQUENCE LISTING (88 sequences)

SEQ ID NO: 1 - protein, 113 aa, Artificial sequence [sequence omitted]

SEQ ID NO: 2 - protein, 107 aa, Artificial sequence [sequence omitted]

SEQ ID NO: 3 - protein, 121 aa, Artificial sequence
  synthetic sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 4 - protein, 108 aa, Artificial sequence
  synthetic sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 5 - protein, 118 aa, Artificial sequence
  synthetic sequence
QVQLQESGPGLVKPSETLSLTCAVSGYSITSDYAWNWIRQPPGKGLEWIGYISYSGSTSYNPSLRSRVTISRDTSKNQFFLKLSSVTAADTAVYYCARRQVGLGFAYWGQGTLVTVSS

SEQ ID NO: 6 - protein, 107 aa, Artificial sequence
  synthetic sequence
DIQMTQSPSSLSASVGDRVTITCKASQDVSTAVAWYQQKPGKAPKLLIYSASYRYTGVPSRFSGSGSGTDFTFTISSLQPEDIATYYCQQHYSTPWTFGQGTKVEIK

SEQ ID NO: 7 - protein, 116 aa, Artificial sequence [sequence omitted]

SEQ ID NO: 8 - protein, 112 aa, Artificial sequence [sequence omitted]

SEQ ID NO: 9 - protein, 107 aa, Artificial sequence
  synthetic sequence
QVQPGGSLRLSCAASGFTFSNFVMHWVRQAPGKGLEWVSSISGSSSSTYYADSVKGRFTISRDNSKNTLYLQMNSLRAEDTAVYYCARYYYASFDYWGQGTLVTVSS

SEQ ID NO: 10 - protein, 108 aa, Artificial sequence
  synthetic sequence
DIELTQPPSVSVAPGQTARISCSGDSLGSKYVYWYQQKPGQAPVVVIYGDSKRPSGIPERFSGSNSGNTATLTISGTQAEDEADYYCGSYTTGAKSHVFGGGTKLTVL

SEQ ID NO: 11 - protein, 122 aa, Artificial sequence [sequence omitted]

SEQ ID NO: 12 - protein, 107 aa, Artificial sequence [sequence omitted]

SEQ ID NO: 13 - protein, 121 aa, Artificial sequence
  synthetic sequence
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 14 - protein, 112 aa, Artificial sequence
  synthetic sequence
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 15 - protein, 113 aa, Artificial sequence
  synthetic sequence
QVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSS

SEQ ID NO: 16 - protein, 112 aa, Artificial sequence
  synthetic sequence
DVVMTQSPLSLPVTLGQPASISCKSSQSLLDSDGKTYLNWFQQRPGQSPNRLIYLVSKLDSGVPDRFSGSGSGTDFTLKISRVEAEDVGVYYCWQGTHFPLTFGGGTKLEIK

SEQ ID NO: 17 - protein, 113 aa, Artificial sequence
  synthetic sequence
QVQLVESGPGLVKPSQSLSITCTVSGFSLSRYSVYWVRQPPGKGLEWLGMMWGGGSTDYNSALKSRLTISKDTSKNQVFLKMNSLRAEDTAMYYCVRTDGDYWGQGTTVTVSS

SEQ ID NO: 18 - protein, 112 aa, Artificial sequence
  synthetic sequence
DLQLTQSPLSLPVSLGDRASISCRSSQSLVNSNGNTYLHWYLQKPGQSPKLLIYKVSNRFSGVPDRFSGSGSGTDFTLKISRVEAEDVGVYYCSQSTHVPWTFGGGTKLEIK

SEQ ID NO: 19 - protein, 127 aa, Artificial sequence [sequence omitted]

SEQ ID NO: 20 - protein, 110 aa, Artificial sequence [sequence omitted]

SEQ ID NO: 21 - protein, 116 aa, Artificial sequence
  synthetic sequence
EVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSS

SEQ ID NO: 22 - protein, 107 aa, Artificial sequence
  synthetic sequence
DIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIK

SEQ ID NO: 23 - protein, 8 aa, Artificial sequence
  synthetic sequence
GITFSNSG

SEQ ID NO: 24 - protein, 8 aa, Artificial sequence
  synthetic sequence
IWYDGSKR

SEQ ID NO: 25 - protein, 8 aa, Artificial sequence
  synthetic sequence
CATNDDYW

SEQ ID NO: 26 - protein, 6 aa, Artificial sequence
  synthetic sequence
QSVSSY

SEQ ID NO: 27 - protein, 3 aa, Artificial sequence
  synthetic sequence
DAS

SEQ ID NO: 28 - protein, 11 aa, Artificial sequence
  synthetic sequence
CQQSSNWPRTF

SEQ ID NO: 29 - protein, 8 aa, Artificial sequence
  synthetic sequence
GFTFSRYW

SEQ ID NO: 30 - protein, 8 aa, Artificial sequence
  synthetic sequence
IKQDGSEK

SEQ ID NO: 31 - protein, 16 aa, Artificial sequence
  synthetic sequence
CAREGGWFGELAFDYW

SEQ ID NO: 32 - protein, 7 aa, Artificial sequence
  synthetic sequence
QRVSSSY

SEQ ID NO: 33 - protein, 3 aa, Artificial sequence
  synthetic sequence
DAS

SEQ ID NO: 34 - protein, 11 aa, Artificial sequence [sequence omitted]

SEQ ID NO: 35 - protein, 9 aa, Artificial sequence
  synthetic sequence
GYSITSDYA

SEQ ID NO: 36 - protein, 7 aa, Artificial sequence
  synthetic sequence
ISYSGST

SEQ ID NO: 37 - protein, 13 aa, Artificial sequence
  synthetic sequence
CARRQVGLGFAYW

SEQ ID NO: 38 - protein, 6 aa, Artificial sequence
  synthetic sequence
QDVSTA

SEQ ID NO: 39 - protein, 3 aa, Artificial sequence
  synthetic sequence
SAS

SEQ ID NO: 40 - protein, 11 aa, Artificial sequence [sequence omitted]

SEQ ID NO: 41 - protein, 8 aa, Artificial sequence
  synthetic sequence
GYTFTSYW

SEQ ID NO: 42 - protein, 8 aa, Artificial sequence
  synthetic sequence
INPRNDYT

SEQ ID NO: 43 - protein, 11 aa, Artificial sequence
  synthetic sequence
CARRDITTFYW

SEQ ID NO: 44 - protein, 12 aa, Artificial sequence
  synthetic sequence
QSVLYSANHKNY

SEQ ID NO: 45 - protein, 3 aa, Artificial sequence
  synthetic sequence
WAS

SEQ ID NO: 46 - protein, 10 aa, Artificial sequence
  synthetic sequence
CHQYLSSWTF

SEQ ID NO: 47 - protein, 8 aa, Artificial sequence
  synthetic sequence
GFTFSNFV

SEQ ID NO: 48 - protein, 8 aa, Artificial sequence
  synthetic sequence
ISGSSSST

SEQ ID NO: 49 - protein, 12 aa, Artificial sequence
  synthetic sequence
CARYYYASFDYW

SEQ ID NO: 50 - protein, 6 aa, Artificial sequence
  synthetic sequence
SLGSKY

SEQ ID NO: 51 - protein, 3 aa, Artificial sequence
  synthetic sequence
GDS

SEQ ID NO: 52 - protein, 13 aa, Artificial sequence
  synthetic sequence
CGSYTTGAKSHVF

SEQ ID NO: 53 - protein, 8 aa, Artificial sequence
  synthetic sequence
GFTFNSFA

SEQ ID NO: 54 - protein, 8 aa, Artificial sequence
  synthetic sequence
ISGSGGGT

SEQ ID NO: 55 - protein, 17 aa, Artificial sequence
  synthetic sequence
CAKDKILWFGEPVFDYW

SEQ ID NO: 56 - protein, 6 aa, Artificial sequence
  synthetic sequence
QSVSSY

SEQ ID NO: 57 - protein, 3 aa, Artificial sequence
  synthetic sequence
DAS

SEQ ID NO: 58 - protein, 11 aa, Artificial sequence [sequence omitted]

SEQ ID NO: 59 - protein, 8 aa, Artificial sequence
  synthetic sequence
GYTFTSYV

SEQ ID NO: 60 - protein, 8 aa, Artificial sequence
  synthetic sequence
INPYNDGT

SEQ ID NO: 61 - protein, 16 aa, Artificial sequence
  synthetic sequence
CARGTYYYGTRVFDYW

SEQ ID NO: 62 - protein, 11 aa, Artificial sequence
  synthetic sequence
KSLQNVNGNTY

SEQ ID NO: 63 - protein, 3 aa, Artificial sequence
  synthetic sequence
RMS

SEQ ID NO: 64 - protein, 11 aa, Artificial sequence
  synthetic sequence
CMQHLEYPITF

SEQ ID NO: 65 - protein, 8 aa, Artificial sequence
  synthetic sequence
GYTFTSYW

SEQ ID NO: 66 - protein, 8 aa, Artificial sequence
  synthetic sequence
IDPSDSET

SEQ ID NO: 67 - protein, 8 aa, Artificial sequence
  synthetic sequence
CARAMGYW

SEQ ID NO: 68 - protein, 11 aa, Artificial sequence
  synthetic sequence
QSLLDSDGKTY

SEQ ID NO: 69 - protein, 3 aa, Artificial sequence
  synthetic sequence
LVS

SEQ ID NO: 70 - protein, 11 aa, Artificial sequence
  synthetic sequence
CWQGTHFPLTF

SEQ ID NO: 71 - protein, 8 aa, Artificial sequence
  synthetic sequence
GFSLSRYS

SEQ ID NO: 72 - protein, 7 aa, Artificial sequence
  synthetic sequence
MWGGGST

SEQ ID NO: 73 - protein, 9 aa, Artificial sequence
  synthetic sequence
CVRTDGDYW

SEQ ID NO: 74 - protein, 11 aa, Artificial sequence
  synthetic sequence
QSLVNSNGNTY

SEQ ID NO: 75 - protein, 3 aa, Artificial sequence
  synthetic sequence
KVS

SEQ ID NO: 76 - protein, 11 aa, Artificial sequence
  synthetic sequence
CSQSTHVPWTF

SEQ ID NO: 77 - protein, 9 aa, Artificial sequence
  synthetic sequence
GGSISGGYG

SEQ ID NO: 78 - protein, 8 aa, Artificial sequence
  synthetic sequence
FYSSSGNT

SEQ ID NO: 79 - protein, 21 aa, Artificial sequence
  synthetic sequence
CVRDRLFSVVGMVYNNWFDVW

SEQ ID NO: 80 - protein, 8 aa, Artificial sequence
  synthetic sequence
TSNIGGYD

SEQ ID NO: 81 - protein, 3 aa, Artificial sequence
  synthetic sequence
DIN

SEQ ID NO: 82 - protein, 13 aa, Artificial sequence
  synthetic sequence
CQSYDSSLNAQVF

SEQ ID NO: 83 - protein, 8 aa, Artificial sequence
  synthetic sequence
GFTFSDAW

SEQ ID NO: 84 - protein, 10 aa, Artificial sequence
  synthetic sequence
IRNKAKNHAT

SEQ ID NO: 85 - protein, 9 aa, Artificial sequence
  synthetic sequence
CGALGLDYW

SEQ ID NO: 86 - protein, 6 aa, Artificial sequence
  synthetic sequence
QEISGY

SEQ ID NO: 87 - protein, 3 aa, Artificial sequence
  synthetic sequence
AAS

SEQ ID NO: 88 - protein, 11 aa, Artificial sequence
  synthetic sequence
CLQYFSYPLTF

(19) United States
(12) Patent Application Publication
(10) Pub. No.: US 2024/0239912 A1
(43) Pub. Date: Jul. 18, 2024

(54) TARGETED REDUCTION OF ACTIVATED IMMUNE CELLS

(71) Applicant: The Board of Trustees of the Leland Stanford Junior University, Stanford, CA (US)

(72) Inventors: William H. Robinson, Palo Alto, CA (US); Tobias V. Lanz, Stanford, CA (US); Samuel J. Rubin, Menlo Park, CA (US)

(21) Appl. No.: 17/917,523

(22) PCT Filed: Apr. 9, 2021
(86) PCT No.: PCT/US21/26690

§ 371 Date: Oct. 6, 2022

Related U.S. Application Data
(60) Provisional application No. 63/008,423, filed on Apr. 10, 2020.

(51) Int. Cl. C07K 16/28; A61P 21/04; A61P 37/00

(52) U.S. Cl. CPC C07K 16/2896; A61P 21/04; A61P 37/00; C07K 16/2803; C07K 16/283; C07K 2317/31; C07K 2317/73; C07K 2317/76

(57) ABSTRACT

The present invention relates to novel bi-specific antigen-binding polypeptides and their preparation and use in the treatment and/or diagnosis of various diseases, and also relates to bi-specific antibody molecules capable of inhibiting immune effector cells and their use in diagnosis and/or treatment of various diseases.

### CROSS-REFERENCE TO RELATED APPLICATIONS

This application is a 371 and claims the benefit of PCT Application No. PCT/US2021/026690, filed Apr. 9, 2021, which claims the benefit of U.S. Provisional Patent Application No. 63/008,423, filed Apr. 10, 2020, which applications are incorporated herein by reference in their entirety.

### STATEMENT REGARDING FEDERALLY SPONSORED RESEARCH

This invention was made with Government support under R01-AR063676 awarded by the National Institutes of Health. The Government has certain rights in the invention.

### INCORPORATION BY REFERENCE OF SEQUENCE LISTING

A Sequence Listing is provided herewith as a Sequence Listing text, S19-151_STAN-1621_SEQ_LIST_ST25, created on Oct. 6, 2022, and having a size of 36,567 bytes. The contents of the Sequence Listing text are incorporated herein by reference in their entirety.

### BACKGROUND

The mammalian immune system serves as a defense against infection, injury and cancer, but it is a double-edged sword. The powerful responses that can be evoked by infection or other undesirable antigenic stimulus can sometimes be evoked by autoantigens, resulting in often long-term damage to otherwise normal tissues.

Activated lymphocytes (B and T cells) are important cellular actors that drive detrimental inflammation in autoimmune and autoinflammatory diseases. Most current treatments for these diseases are broadly immunosuppressive. More specific therapies, tailored to inhibit activated lymphocyte subsets, promise to be more effective with less side effects. Lymphocytes express immunomodulatory receptors on their cell surface that either activate or inhibit the cell. Blocking inhibitory receptors (so-called “checkpoints”) have been exploited successfully in cancer therapy. Activation of these inhibitory receptors would suppress lymphocyte activation and could be exploited therapeutically for autoimmune diseases. Many of these inhibitory receptors attract tyrosine phosphatases, often via ITIM motifs located at the receptors' intracellular domains, which disrupt activating signaling cascades by inhibiting phosphorylation. Expression of cell surface receptors is tightly regulated and restricted to certain lymphocyte subsets. Targeting of specific pathogenic cellular subsets can be achieved by selecting the right set of receptors.

B cells can be characterized by their ability to differentiate into plasmablasts and plasma cells and to produce antibodies. In addition, B cells have important functions in the pathogenesis of autoimmune diseases, including autoimmune rheumatic diseases. In addition to producing autoantibodies, B cells contribute to autoimmunity by serving as professional antigen-presenting cells (APCs), producing cytokines, and through additional mechanisms. B cell activation and effector functions are regulated by immune checkpoints, including both activating and inhibitory checkpoint receptors that contribute to the regulation of B cell tolerance, activation, antigen presentation, T cell help, class switching, antibody production and cytokine production. The various activating checkpoint receptors include B cell activating receptors that engage with cognate receptors on T cells or other cells, as well as Toll-like receptors that can provide dual stimulation to B cells via co-engagement with the B cell receptor. Furthermore, various inhibitory checkpoint receptors, including B cell inhibitory receptors, have important functions in regulating B cell development, activation and effector functions. Therapeutically targeting B cell checkpoints represents a promising strategy for the treatment of a variety of autoimmune rheumatic diseases.

Although B cell-depleting therapies that, for example, target CD19 or CD20, are effective in treating multiple autoimmune diseases, these therapies also result in immune deficits. Specifically, anti-CD20 B cell-depleting therapies confer increased susceptibility to infections and in a subset of patients result in chronically low serum antibody titers that further increase the risk of infection. Furthermore, B cell-depleted patients have reduced responses to vaccination. As a result of the limitations and complications of antiCD20 B cell-depleting antibody therapeutics, new types of treatments that either deplete specific subsets of pathogenic B cells or modulate B cell activation and function in more precise manners are needed for the treatment of autoimmunity.

The complexity of the immune cell populations and interactions requires a specific and targeted approach for therapeutics. The present disclosure addresses this need.

### SUMMARY OF THE INVENTION

Compositions and methods are provided for targeted reduction of activated immune cells, where reduction may decrease activity of viable cells, or may decrease the number of targeted viable activated cells. The compositions and methods find use, for example, in the treatment of inflammatory diseases, including without limitation autoimmune diseases.

Multi-specific antibodies, e.g. bi-specific antibodies, are provided that comprise at least two different antigen binding regions (ABR), where an antigen binding region usually combines two antibody variable region sequences, which may be provided as one or two polypeptide chains, and may be joined to one or more constant region sequences. A first ABR specifically binds to an extracellular domain of an inhibitory receptor expressed on activated immune cells, e.g. activated lymphocytes. Inhibitory receptors include, without limitation, CD22, CD32b, CD95, BTLA, CD72, LAIR1, CD85j, LAG-3, PD1, TIGIT, CTLA4, TIM3, VISTA, 2B4, CD5, 4-1BB, CD2, CD49b, ICOS, TIM1, OX40, CD357, CD30, etc.

A second ABR specifically binds to a member of the T cell receptor (TCR) or B cell receptor (BCR) complex, a molecule associated with a member of the T cell receptor (TCR) or B cell receptor (BCR) complex, a molecule that comes into proximity to the TCR or the BCR during certain stages of lymphocyte activation, an activating co-receptor expressed on lymphocytes, a receptor with an intrinsic intracellular tyrosine-kinase activity or with the ability to recruit tyrosine-kinases to its intracellular domain, or a subset-specific receptor, which may be referred to as a targeting marker. Markers of interest for this purpose include, without limitation, CD3, CD4, CD8, CD79b, CD19, CD20, CD38, CD138, CD95, CD93, CD69, CD30, PD-1, CD40, BCMA, GPRC5D, BTLA, LAG-3, CD70, PLD4, CD27, CD80, CD86, CD226, 4-1BB, CD2, CD49b, ICOS, TIM1, OX40, CD357, CD30, etc.

Binding of both ABRs on the cell surface creates proximity between the inhibitory receptor's intracellular domain and the activating signaling cascade, resulting in inhibition of the cell and reducing activity of the cell. Either the inhibitory molecule or the targeting marker (second molecule) or both are cell subset-specific, ensuring that inhibition is restricted to the targeted lymphocyte subset. The multi-specific antibodies are usually bi-specific antibodies. Various formats of bi-specific antibodies are within the ambit of the invention, including without limitation single chain polypeptides, two chain polypeptides, three chain polypeptides, four chain polypeptides, and multiples thereof. In some embodiments the antibodies are bivalent. In some embodiments the antibodies are tetravalent. In some embodiments the antibodies are bivalent with a low amount of artificial mutations and low potential for immunogenicity, including without limitation a knob-in-hole format and derivatives of the knob-in-hole format.

In some embodiments the activated lymphocyte is a B cell. In such embodiments, the first ABR may bind, for example, to an inhibitory receptor selected from CD22, CD32b, CD95, BTLA, CD72, LAIR1, CD85j and LAG-3. In certain embodiments, the inhibitory receptor is CD32b. In such embodiments the second ABR may bind to, for example, CD38, CD138, CD30, CD95, CD93, BCMA, GPRC5D, PD-1, BTLA, LAG-3, CD70 and PLD4. In some embodiments the second ABR binds to CD38. In some embodiments, a bivalent or tetravalent bi-specific antibody is provided that selectively binds to CD32b; and to CD38, which can target plasmablasts and plasma cells. In some embodiments a bivalent or tetravalent bi-specific antibody is provided that selectively binds to CD38 and CD22, which can target plasma cells. In some embodiments, a bivalent or tetravalent bi-specific antibody is provided that selectively binds to CD32b; and to PD-1, which can target plasmablasts and plasma cells. In some embodiments a bi-specific antibody is provided that selectively binds to PD1 and to CD38.

In some embodiments the activated lymphocyte is a T cell. In such embodiments the first ABR may bind to an inhibitory receptor selected from PD-1, BTLA, CD5, TIGIT, CTLA4, VISTA, TIM3, LAG3, and 2B4. In some embodiments the second ABR may bind to, for example, CD3, CD4, CD8, CD38, CD69, CD30 and PD-1.

Bi-specific antibodies of interest include, for example, antibodies that bind to the combinations of CD19 and CD22; CD19 and CD32b; CD19 and PD-1; CD20 and CD32b; CD79b and CD22; CD79b and CD30; CD79b and PD-1; CD69 and CD22; CD69 and PD-1; CD69 and CD32b; CD69 and PD-1; CD69 and TIGIT; CD69 and PD-L1; PD-1 and CD32b; PD-1 and CD30; CD-1 and CD80; CD40 and CD22; and CD40 and PD-1. The ABR sequences may utilize a known antibody, e.g. an antibody in clinical use or clinical trials. Alternatively antibodies can be developed for this purpose.

In some embodiments, a multi-specific antibody of the invention comprises at least one variable region sequence from the sequences provided in Table 2A, e.g. the polypeptides of SEQ ID NO:1-SEQ ID NO:22. In some embodiments the multi-specific antibody comprises a pair of variable region sequences, e.g. SEQ ID NO:1 and 2; SEQ ID NO:3 and 4; SEQ ID NO:5 and 6; SEQ ID NO:7 and 8; SEQ ID NO:9 and 10; SEQ ID NO:11 and 12; SEQ ID NO:13 and 14; SEQ ID NO:15 and 16; SEQ ID NO: 17 and 18; SEQ ID NO: 19 and 20; SEQ ID NO:21 and 22. In some embodiments the multi-specific antibody comprises a variable region sequence comprising a VH CDR1, CDR2 and CDR3 sequence of SEQ ID NO:1 and a VL CDR1, CDR2 and CDR3 sequence of SEQ ID NO:2; a VH CDR1, CDR2 and CDR3 sequence of SEQ ID NO:3 and a VL CDR1, CDR2 and CDR3 sequence of SEQ ID NO:4; a VH CDR1, CDR2 and CDR3 sequence of SEQ ID NO:5 and a VL CDR1, CDR2 and CDR3 sequence of SEQ ID NO:6; a VH CDR1, CDR2 and CDR3 sequence of SEQ ID NO:7 and a VL CDR1, CDR2 and CDR3 sequence of SEQ ID NO:8; a VH CDR1, CDR2 and CDR3 sequence of SEQ ID NO:9 and a VL CDR1, CDR2 and CDR3 sequence of SEQ ID NO:10; a VH CDR1, CDR2 and CDR3 sequence of SEQ ID NO:11 and a VL CDR1, CDR2 and CDR3 sequence of SEQ ID NO:12; a VH CDR1, CDR2 and CDR3 sequence of SEQ ID NO:13 and a VL CDR1, CDR2 and CDR3 sequence of SEQ ID NO:14; a VH CDR1, CDR2 and CDR3 sequence of SEQ ID NO:15 and a VL CDR1, CDR2 and CDR3 sequence of SEQ ID NO:16; a VH CDR1, CDR2 and CDR3 sequence of SEQ ID NO:17 and a VL CDR1, CDR2 and CDR3 sequence of SEQ ID NO:18; a VH CDR1, CDR2 and CDR3 sequence of SEQ ID NO:19 and a VL CDR1, CDR2 and CDR3 sequence of SEQ ID NO:20; a VH CDR1, CDR2 and CDR3 sequence of SEQ ID NO:21 and a VL CDR1, CDR2 and CDR3 sequence of SEQ ID NO:22. Exemplary CDR sequences are provided in Table 2B.

In other embodiments, pharmaceutical compositions are provided, comprising at least a bi-specific antibody as described herein; and a pharmaceutically acceptable excipient. The composition may be lyophilized, suspended in solution, etc. and may be provided in a unit dose formulation.

In some embodiments, a method is provided for treatment of inflammatory disease, the method comprising administering to an individual in need thereof an effective dose of a bi-specific antibody of the disclosure. In some embodiments the disease is an autoimmune disease. In some embodiments the autoimmune disease is a rheumatoid autoimmune disease, for example rheumatoid arthritis; myasthenia gravis; systemic lupus erythematosis (SLE), ankylosing spondylitis (AS); psoriatic arthritis (PsA); scleroderma; Sjogren's syndrome, IgG4-related disease, etc. In some embodiments the autoimmune disease is a demyelinating autoimmune disease, e.g. neuromyelitis optica, multiple sclerosis, Guillain-Barre syndrome, etc. In some embodiments the autoimmune disease is a metabolic disease, e.g. IDDM, Type 2 diabetes, NAFLD, and the like.

In other embodiments, a method is provided for the production of a bi-specific antibody of the present invention comprising expressing the antibody sequences, e.g. one or more light chain encoding sequences, one or more heavy chain encoding sequences, in a single host cell. In various embodiments, the host cell may be a prokaryotic or an eukaryotic cell, such as a mammalian cell.

### BRIEF DESCRIPTION OF THE DRAWINGS

FIG. 1: Schematic showing a bi-specific antibody binding a marker that defines activated B cell subsets (targeting molecule) as well as an inhibitory cell surface receptor, thereby creating proximity between both cell surface receptors.

FIG. 2: Screening data: (A) flow cytometry gating scheme. CD19⁺ B cells are gated on CD20^{low} B cells and CD27^{high} as well as CD38^{high} B cells, encompassing activated plasmablasts and plasma cells. (B) Expression data of selected inhibitory cell surface receptors, including CD38, CD32b, PD-1, and CD22. Expression levels (mean fluorescence intensity, MFI) are shown for IgD CD27 double negative B cells, naïve B cells, unswitched and switched memory B cells as well as plasmablasts and plasma cells.

FIG. 3: (A) List of tested combinations of targeting molecules (activation markers) and inhibitory receptors. (B) List of bi-specific formats used for bi-specific screening. Unless otherwise specified, the IgG(H)-scFV (Morrison) was used in experiments.

FIG. 4: Survival data of B cells incubated with different bi-specific antibody molecules and with monospecific anti-CD38 antibody. (A) overall survival of B cells, and (B) survival of plasmablasts.

FIG. 5. Cell surface expression of activation markers on B cells after different stimulations with several bi-specific molecules and anti-CD38. (A) Cell surface expression of the early activation marker CD69, (B) cell surface expression of HLA-DR.

FIG. 6. B cell secretion of the inflammatory cytokines TNF-alpha and Interleukin-6 (IL-6) upon incubation with bi-specific molecules CD38×CD32b and CD79×CD32b as well as mono-specific anti-CD38.

FIG. 7. Activation (ITIM phosphorylation) of CD32b by the bi-specific antibody CD38×CD32b vs. isotype. Western blots for CD32b and phospho-CD32b are shown from B cell lysate upon stimulation with the bi-specific compound as well as the additional inflammatory stimulus.

FIG. 8: CD38×CD32b bi-specific antibody inhibits CD38 enzymatic activity. Apigenin=small molecule inhibitor of CD38 NADase.

FIG. 9. Expression Levels of Suppressive Receptors in T cell Subsets. (published data, see: Murphy K A, Bhamidipati K, Rubin S J S, Kipp L, Robinson W H, Lanz T V. “Immunomodulatory Receptors Are Differentially Expressed in B and T Cell Subsets Relevant to Autoimmune Disease” Clinical Immunology 209 (Oct. 25, 2019): 108276. doi:10.1016/j.clim.2019.108276.)

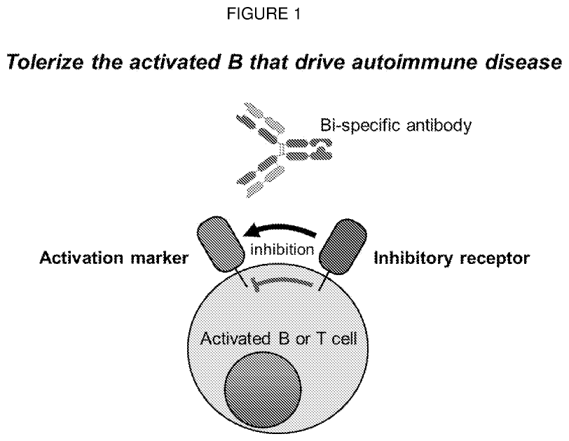

### DETAILED DESCRIPTION OF INVENTION

To facilitate an understanding of the invention, a number of terms are defined below.

Before the present active agents and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

### Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

Inhibitory markers. As used herein, inhibitory markers, or inhibitory receptors, are proteins expressed on activated immune cells, particularly activated B and T lymphocytes, that act to decrease immune responsiveness toward an antigen recognized by the lymphocyte.

Such receptors may include, without limitation, receptors expressed on B cells. For example, CD22 is a pan B cell marker except plasmablasts; CD32b is a pan-B cell marker; CD95 is expressed on plasmablasts and plasma cells; PD-1 is expressed on plasmablasts and plasma cells; BTLA is expressed on plasma cells; CD72 is expressed on plasma cells; LAIR1 is expressed on plasma cells; LAG-3 is expressed on plasma cells; CD85j is a pan-B cell marker.

CD22 is a sugar binding transmembrane protein, which specifically binds sialic acid with an immunoglobulin (Ig) domain located at its N-terminus. The presence of Ig domains makes CD22 a member of the immunoglobulin superfamily. During B cell development and maturation, inhibitory checkpoints balance activating signals to ensure central tolerance. CD22 is important for regulating B cell activation in response to self-antigens. The receptor is constitutively associated with the BCR and contains three cytoplasmic immunoreceptor tyrosine-based inhibition motifs (ITIMs), which are phosphorylated upon ligand binding in conjunction with BCR-antigen binding. Subsequently, recruitment of the phosphatases SHIP1 and SHP1 inhibits signaling downstream of the BCR. This inhibition is thought to modulate the BCR activation threshold to prevent B cell activation in response to self-antigens.

CD32b (FcγRIIb, coded by FCGR2B gene) is a low affinity inhibitory receptor for the Fc region of immunoglobulin gamma (IgG). FcγRIIb participates in the phagocytosis of immune complexes and in the regulation of antibody production by B lymphocytes. The receptor inhibits the functions of activating FcγRs, such as phagocytosis and pro-inflammatory cytokine release, mainly by clustering of FcγRIIb with different activating FcγRs or with the BCR by immune complexes. The phosphorylated ITIM of FcγRIIb recruits the inositol phosphatases SHIP1 and SHIP2 as well as the tyrosine phosphatases SHP1 and SHP2. SHIP1 and SHIP2 inhibit Ras activation, downregulate MAPK activity and reduce PLCy function and lead to decreased activation of PKC. Inhibition of the MAP kinase pathway, together with the anti-apoptotic kinase Akt can negatively affect proliferation and survival of the cells. SHP1 and SHP2 directly interfere in phosphorylation signaling cascades downstream of BCR and CD40. FcγRIIb regulates B cell activation by increasing the BCR activation threshold and suppressing B cell mediated antigen presentation to T cells through the ITIM-dependent inhibitory mechanism. Ligation of FcγRIIb on B cells downregulates antibody production, prevents the membrane organization of BCR and CD19 and promotes apoptosis. The low-affinity immunoglobulin-γ FcγRIIb is the only inhibitory FcR and the only FcR expressed by B cells; this molecule is crucial for restricting antibody-mediated immune responses. This process is thought to modulate immune responses when antigens saturated with IgG antibodies in immune complexes trigger FcγRIIb in conjunction with the BCR60. As a result, FcγRIIb is thought to help prevent autoimmunity, and mutations at its genetic locus have been associated with RA, SLE and multiple sclerosis in human genome-wide association studies and mouse studies.

Antibodies specific for human CD32b are known in the art and are clinically applicable, for example see Veri, Maria-Concetta et al. “Monoclonal antibodies capable of discriminating the human inhibitory Fcgamma-receptor IIB (CD32B) from the activating Fcgamma-receptor IIA (CD32A): biochemical, biological and functional characterization.” Immunology vol. 121,3 (2007): 392-404. doi:10.1111/j.1365-2567.2007.02588.x; Rankin et al. Blood (2006) 108 (7): 2384-2391, “CD32B, the human inhibitory Fc-γ receptor IIB, as a target for monoclonal antibody therapy of B-cell lymphoma” for the antibodies ch2B6 and hu2B6-3.5; Cox et al. Cell 27:473-488 (2015) for the antibody BI-1206; etc. Alternatively, antibodies can be generated and screened for this purpose.

CD95 (Fas/APO-1/TNFRSF6), is a cell surface protein that belongs to the tumor necrosis factor receptor family, can mediate apoptosis when bound to its natural ligand, CD95L (CD178/TNFSF6) or stimulated with agonistic antibodies. It is ubiquitously expressed in the body, but is particularly abundant in the thymus, liver, heart, and kidney. CD95L is predominantly expressed in activated T lymphocytes and natural killer cells, and is constitutively expressed in tissues of ‘immune-privilege sites’ such as the testis and eye.

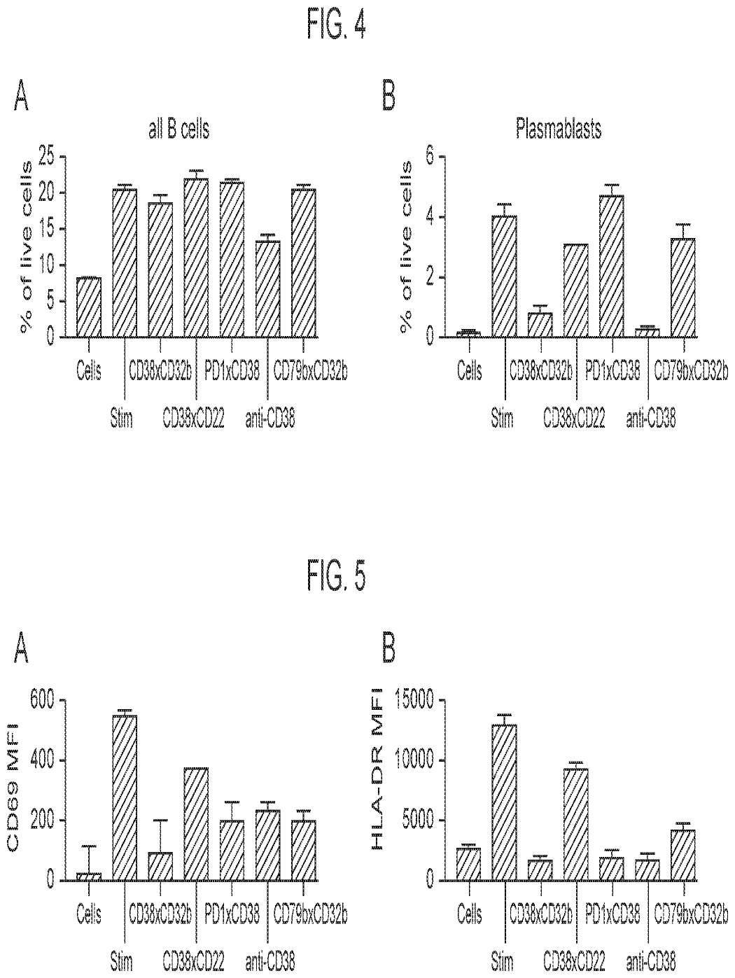

Programmed cell death protein 1, also known as PD-1 and CD279 (cluster of differentiation 279), is a protein on the surface of cells that has a role in regulating the immune system's response to the cells of the human body by down-regulating the immune system and promoting self-tolerance by suppressing T cell inflammatory activity. One major role of PD1 is to limit the activity of T cells in peripheral tissues at the time of an inflammatory response to infection and to limit autoimmunity. PD1 expression is induced when immune cells become activated. The two ligands for PD1 are PD1 ligand 1 (PDL1; also known as B7-H1 and CD274) and PDL2 (also known as B7-DC and CD273). An bi-specific antibody with an inhibitory anti-PD1 specificity has been used to generate the evidence shown in FIGS. 4 and 5. It is likely, that efficacy of plasmablast inhibition will be increased by using a non-blocking anti-PD1 binding molecule.

B- and T-lymphocyte attenuator (BTLA) is an immune-regulatory receptor, similar to CTLA-4 and PD-1, and is mainly expressed on B-, T-, and all mature lymphocyte cells. BTLA is a type I transmembrane glycoprotein comprising 289 amino acids. Its protein structure is similar to those of CTLA-4 and PD-1 and includes an extracellular domain, transmembrane domain, and cytoplasmic domain. The cytoplasmic domain contains three conserved signals: a growth factor receptor-bound protein-2 (Grb-2) recognition motif, an immunoreceptor tyrosine-based inhibitory motif (ITIM), and an immunoreceptor tyrosine-based switch motif (ITSM). ITIM is present in many inhibitory receptors, binding and activating the tyrosine phosphatases SHP-1 and SHP-2, which dephosphorylate tyrosine and inhibit protein tyrosine kinase (PTK)-dependent cell activation. The Grb-2 recognition motif recognizes the Grb-2 protein, recruits the PI3K protein subunit p85, and stimulates the PI3K signaling pathway, promoting cell proliferation and survival. Thus, the BTLA molecule exerts bidirectional regulatory effects.

CD72, a type II membrane protein expressed mostly in B cells, contains a C-type lectin-like domain (CTLD) in the extracellular region and an immunoreceptor tyrosine-based inhibition motif (ITIM) in the cytoplasmic region. By recruiting SH2 domain-containing phosphatase 1 (SHP-1) to the phosphorylated ITIM, CD72 negatively regulates B cell antigen receptor (BCR) signaling.

Leukocyte-associated Ig-like receptor-1 (LAIR-1) belongs to the family of immunoreceptor tyrosine-based inhibitory motif-bearing receptors and is expressed on the majority of peripheral mononuclear cells, including NK cells, T cells, B cells, monocytes, and dendritic cells. LAIR-1 is expressed from early on during B cell differentiation, but is absent on approximately half of the memory B cells, and all germinal center B cells, plasmablasts, and terminally differentiated plasma cells. It can function as a negative regulator of BCR-mediated signaling, since simultaneous cross-linking of LAIR-1.

LAG3 is a CD4 homolog. LAG3's main ligand is MHC class II, to which it binds with higher affinity than CD4. The protein negatively regulates cellular proliferation, activation, and homeostasis of immune cells, in a similar fashion to CTLA-4 and PD-1 and has been reported to play a role in Treg suppressive function.

CD85j (LILRB1) is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response.

TABLE 1
Targeting Molecules

| Marker | Cell type |
|---|---|
| CD38 | plasmablast and plasma cell |
| CD138 | plasma cell |
| CD30 | plasma cell |
| CD95* | plasmablast and plasma cell |
| CD93 | plasma cell |
| BCMA | plasmablast and plasma cell |
| GPRC5C | plasma cell |
| GPRC5D | plasma cell |
| PD-1* | plasmablast and plasma cell |
| BTLA* | plasma cell |
| LAG-3* | plasma cell |
| CD70 | memory cell and ASC |
| PLD4 | plasma cell |

Targeting marker. As used herein, a targeting marker is a protein expressed, at least although not necessarily exclusively, on activated immune cells, particularly activated lymphocytes such as B cells and T cells. In some embodiments the targeting marker is a member of the T cell receptor (TCR) or B cell receptor (BCR) complex, a molecule associated with a member of the T cell receptor (TCR) or B cell receptor (BCR) complex, an activating co-receptor expressed on lymphocytes, or a subset-specific receptor. The targeting marker may also be an inhibitory marker, although at least two distinct proteins are recognized by the bi-specific antibodies.

Targeting markers of interest include, without limitation, CD3, CD4, CD8, CD79b, CD19, CD20, CD38, CD138, CD95, CD93, CD69, CD30, PD-1, CD40, BCMA, GPRC5D, BTLA, LAG-3, and CD70, PLD4, CC27, CD80, CD86, CD226, 4-1BB, CD2, CD49b, ICOS, TIM1, OX40, CD357, and CD30.

For example, the T cell markers CD3, CD4 and CD8 are well-known in the art as markers of T cells (CD3) and T cell subsets (CD4 and CD8). Antibodies specific for these proteins are known and used in the art. For example, antibodies specific for human CD4 include keliximab, clenoliximab, MAX.16H5, and 412W94. IT1208 is a defucosylated humanized anti-CD4 monoclonal antibody. Antibodies specific for CD3 include OKT3 and variants thereof, e.g. anti-huOKT3γ1 (ala-ala); muromonab, otelixizumab, teplizumab and visilizumab.

CD19 is a biomarker for normal and neoplastic B cells, as well as follicular dendritic cells. CD19 is critically involved in establishing intrinsic B cell signaling thresholds through modulating both B cell receptor-dependent and independent signaling. Blinatumomab, Coltuximabravtansine, MOR208, MEDI-551, Denintuzumabmafodotin, Merck patent anti-CD19, Taplitumomabpaptox, XmAb 5871, MDX-1342, AFM11 are examples of antibodies suitable for clinical use.

CD20 is a B-lymphocyte-specific membrane protein that plays a role in the regulation of cellular calcium influx necessary for the development, differentiation, and activation of B-lymphocytes. It is the target of monoclonal antibodies rituximab, ocrelizumab, obinutuzumab, ofatumumab, ibritumomab tiuxetan, tositumomab, and ublituximab, which are all active agents in the treatment of all B cell lymphomas, leukemias, and B cell-mediated autoimmune diseases. Additional anti-CD20 antibody therapeutics under development include ocaratuzumab, and veltuzumab.

CD79b is required in cooperation with CD79A for initiation of the signal transduction cascade activated by the B-cell antigen receptor complex (BCR) which leads to internalization of the complex, trafficking to late endosomes and antigen presentation. It enhances phosphorylation of CD79A, possibly by recruiting kinases which phosphorylate CD79A or by recruiting proteins which bind to CD79A and protect it from dephosphorylation. The antibody polatuzumab is specific for CD79b and is in clinical trials.

CD69 is a human transmembrane C-Type lectin protein. It is an early activation marker that is expressed in hematopoietic stem cells, T cells, and many other cell types in the immune system. It is also implicated in T cell differentiation as well as lymphocyte retention in lymphoid organs.

CD38, also known as cyclic ADP ribose hydrolase is a glycoprotein found on the surface of many immune cells (white blood cells), including CD4+, CD8+, B lymphocytes and natural killer cells. CD38 also functions in cell adhesion, signal transduction and calcium signaling and is known to co-localize with the immunological synapse during B and T cell activation. Antibodies specific for human CD38 include, for example, MOR03087, AMG 424, daratumumab, and SAR650984.

CD40 is a costimulatory protein found on antigen-presenting cells and is required for their activation. The binding of CD154 (CD40L) on TH cells to CD40 activates antigen presenting cells and induces a variety of downstream effects. Antibodies include, for example, CD40 Agonistic Monoclonal Antibody APX005M, BI 655064, e.g. with an engineered Fc region or lacking an Fc region to minimize toxicity.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g. mouse, rat, etc.

“Antibody-dependent cell-mediated cytotoxicity” and “ADCC” refer to a cell-mediated reaction in which nonspecific cytotoxic cells that express Fc receptors, such as natural killer cells, neutrophils, and macrophages, recognize bound antibody on a target cell and cause lysis of the target cell. ADCC activity may be assessed using methods, such as those described in U.S. Pat. No. 5,821,337. ADCP refers to antibody dependent cell-mediated phagocytosis.

“Effector cells” are leukocytes which express one or more constant region receptors and perform effector functions.

A “cytokine” is a protein released by one cell to act on another cell as an intercellular mediator.

“Non-immunogenic” refers to a material that does not initiate, provoke or enhance an immune response where the immune response includes the adaptive and/or innate immune responses.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A pharmaceutical formulation is a composition comprising different chemical substances including but not limited to active drugs, excipients, etc. which are combined and formulated to produce a final medicinal product for the treatment of humans or other organisms.

A sterile formulation is a formulation substantially free of living germs or microorganisms.

A therapeutically effective amount is that mass of an active drug in a formulation, and the frequency of administration of a formulation, that results in the prevention of the development of symptoms, prevention of development of markers or signs of a disease, prevention of the development of tissue or organ damage, prevention of the progression of a disease, reduction in the severity of a disease, or treatment of disease symptoms as defined above.

It is within the level of skill of a clinician to determine the preferred route of administration and the corresponding dosage form and amount, as well as the dosing regimen, i.e., the frequency of dosing. Such information may be obtained in a straightforward manner in accordance with the teachings and guidelines contained in the instant specification taken in light of the knowledge and skill of the artisan. The results that are obtained can also be correlated with data from corresponding evaluations of an approved product in the same assays.

Dose range for an agent is the range of the mass of active drug in, and frequency of administration of, a formulation which results in the prevention of the development of symptoms, prevention of the development of a disease, prevention of development of abnormal markers or signs of a disease, prevention of the development of tissue or organ damage, prevention of the progression of a disease, reduction in the severity of a disease, or treatment of disease symptoms as defined above.

Regimen means dose, frequency of administration, for example twice-per day, daily, weekly, bi-weekly etc., and duration of treatment, for example one day, several days, one week, several weeks, one month, several months, one year, several years, etc.

Unit doses (also called dosage forms) are essentially pharmaceutical products in the form in which they are marketed for use, typically involving a mixture of active drug components and nondrug components (excipients), along with other non-reusable material that may not be considered either ingredient or packaging (such as a capsule shell, for example). Depending on the context, multi(ple) unit dose can refer to distinct drug products packaged together, or to a single drug product containing multiple drugs and/or doses. The term dosage form can also sometimes refer only to the chemical formulation of a drug product's constituent drug substance(s) and any blends involved.

A dose pack is a premeasured amount of drug to be dispensed to a patient in a set or variable dose and in a package including but not limited to a blister pack or other series of container for the purpose of facilitating a dose regimen. A dose pack can be used to facilitate delivery of an initial and/or loading dose to an individual, followed by a maintenance dose.

An excipient is generally a pharmacologically inactive substance formulated with the active pharmaceutical ingredient (“API”) of a medication. Excipients are commonly used to bulk up formulations that contain potent active ingredients (thus often referred to as “bulking agents,” “fillers,” or “diluents”), to allow convenient and accurate dispensation of a drug substance when producing a dosage form. They also can serve various therapeutic-enhancing purposes, such as facilitating drug absorption or solubility, or other pharmacokinetic considerations.

A “marker of inflammation” (also referred to herein as biomarkers of inflammation) is an objectively measured characteristic that reflects the presence of inflammation in a pre-disease state, or disease state including but not limited to molecular, biochemical, imaging, or gross physical measurements. As used herein, “markers of inflammation” include inflammatory markers, metabolic markers, imaging markers, biochemical markers, genetic markers, proteomic markers, gene expression markers, and other markers that can be used to assess inflammation within an individual. The measurement of abnormal markers in an individual identifies that individual as being at increased risk for development of, in the pre-clinical phases of, in the early-stages of, or having an established, inflammatory disease or disease associated with inflammation.

Molecular marker(s) of inflammation (also referred to herein as a “biomarker” or “biomarkers of inflammation”) are molecules obtained from tissue (e.g., blood) samples of a patient which indicate the presence of inflammation. Nonlimiting example of such markers can include, for example, C-reactive protein, a cytokine, an antibody, a DNA sequence, an RNA sequence, a cartilage marker, a metabolic marker, a bone marker, or combinations thereof. Molecular markers of inflammation include biochemical markers.

Imaging marker(s) of inflammation (also referred to herein as an “imaging marker” or “imaging biomarkers”) are markers that measure or otherwise determine the presence of inflammation through use of an imaging modality, including but not limited to ultrasound, radiography, computerized tomography, magnetic resonance imaging, or nuclear medical scanning.

Biochemical marker(s) (also referred to herein as a “molecular marker”) are biologic substances that are measured in blood or other tissue as a biomarker. Biological biomarkers of interest include without limitation proteins, nucleic acids, metabolites, fatty acids, peptides, and the like.

“Inflammatory markers” (also referred to herein as an “inflammatory biomarkers”) are biomarkers indicating an inflamed state. Inflammatory biomarkers of interest include without limitation cytokines, chemokines, high sensitivity C-reactive protein (hs-CRP), erythrocyte sedimentation rate (ESR), expression of mRNA encoding inflammatory mediators, inflammatory cells, imaging biomarkers demonstrating inflammation, and other markers indicative of inflammation.

A reference range is defined as the set of values within which 95 percent of the normal population falls. It typically refers to the value or level of a marker (as termed herein as a “marker” or “biomarker”, and examples of such markers include but are not limited to inflammatory markers, metabolic markers, imaging markers, biochemical markers, clinical markers, radiographic markers, and other biomarkers. If the value or level of a marker in an individual patient is outside the set of values or levels within which 95 percent of the normal population falls, then the marker is considered to exhibit an abnormal level in that patient (e.g. that patient is determined to have an “abnormal marker”). In some embodiments, if the value or level of an inflammatory marker in an individual patient is outside the set of values or levels within which 95 percent of the normal population falls, then the inflammatory marker is considered to exhibit an abnormal level in that patient (e.g. that patient is determined to have an “abnormal inflammatory marker”). In some embodiments, if the value or level of a metabolic marker (also termed a “metabolic disease marker”) in an individual patient is outside the set of values or levels within which 95 percent of the normal population falls, then the metabolic marker is considered to exhibit an abnormal level in that patient (e.g. that patient is determined to have an “abnormal metabolic marker”). In some embodiments, if the result of an imaging marker in an individual patient is outside the range of variation of the same imaging marker observed within 95 percent of the normal population, then the imaging marker is considered to exhibit an abnormal result (e.g. it is an “abnormal imaging marker”). In some embodiments, if the result of a clinical marker in an individual patient is outside the range of variation of the same clinical marker observed within 95 percent of the normal population, then the clinical marker is considered to exhibit an abnormal result (e.g. it is an “abnormal clinical marker”). The measurement of abnormal markers in an individual identifies that individual as being at increased risk for development of, in the pre-clinical phases of, in the early-stages of, or having an established, inflammatory disease or disease associated with inflammation.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass humans having pre- or early-stage inflammatory disease. Subjects may be human, but also include other mammals, particularly those mammals useful as laboratory models for human disease, e.g. mouse, rat, cats, dogs, horses, etc.

The expression “body fluid” as used herein in intended to include all of those accessible body fluids usable as clinical specimens which may contain a compound being tested for in sufficient concentration in said fluid to be within the limits of detection of the test device or assay being used. Body fluids will thus include whole blood, serum, plasma, urine, cerebrospinal fluid, synovial fluid, and interstitial and other extracellular fluids, particularly synovial fluid of affected joints. In some embodiments a body fluid used for determination of an abnormal marker of early-stage inflammation is a synovial fluid from a joint suspected of being involved in early arthritis. In other embodiments a body fluid used for marker determination is systemic, e.g. blood, urine, etc.

“Homology” between two sequences is determined by sequence identity. If two sequences, which are to be compared with each other, differ in length, sequence identity preferably relates to the percentage of the nucleotide residues of the shorter sequence which are identical with the nucleotide residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98, appended examples and http://workbench.sdsc.edu/). For this purpose, the “default” parameter settings may be used.

“Variant” refers to polypeptides having amino acid sequences that differ to some extent from a native sequence polypeptide. Ordinarily, amino acid sequence variants will possess at least about 80% sequence identity, more preferably, at least about 90% homologous by sequence. The amino acid sequence variants may possess substitutions, deletions, and/or insertions at certain positions within the reference amino acid sequence.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

The term “host cell” (or “recombinant host cell”), as used herein, is intended to refer to a cell that has been genetically altered, or is capable of being genetically altered by introduction of an exogenous polynucleotide, such as a recombinant plasmid or vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

“Binding affinity” generally refers to the strength of the sum total of noncovalent interactions between a single binding site of a molecule (e.g., an antibody or other binding molecule) and its binding partner (e.g., an antigen or receptor). The affinity of a molecule X for its partner Y can generally be represented by the dissociation constant (Kd). Affinity can be measured by common methods known in the art, including those described herein. Low-affinity antibodies bind antigen (or receptor) weakly and tend to dissociate readily, whereas high-affinity antibodies bind antigen (or receptor) more tightly and remain bound longer.

Unless specifically indicated to the contrary, the term “conjugate” as described and claimed herein is defined as a heterogeneous molecule formed by the covalent attachment of one or more antibody fragment(s) to one or more additional molecules, such as polymer molecule(s), labels, cytotoxic agents, etc. For example a polymer may be water soluble, i.e. soluble in physiological fluids such as blood, and wherein the heterogeneous molecule is free of any structured aggregate. A conjugate of interest is PEG. The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

### Proteins

The present disclosure provides for bi-specific antibodies that bind to and inhibit activated immune cells. Each ABR may utilize known CDR sequences, or may utilize CDRs selected from an immunization, a phage, bacterial or mammalian display or from other methods suitable to generate new antibodies. A suitable antibody may be selected for development and use, including without limitation use as a bi-specific antibody. Determination of affinity for a candidate protein can be performed using methods known in the art, e.g. Biacore measurements, etc. Members of the antibody family may have an affinity for the cognate antigen with a Kd of from about 10-7 to around about 10-11, including without limitation: from about 10-7 to around about 10⁻¹⁰; from about 10⁻⁷ to around about 10⁻⁹; from about 10⁻⁷ to around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁹ to around about 10⁻¹⁰; or any value within these ranges. The affinity selection may be confirmed with a biological assessment for inhibition of B or of T cells in, for example, and in vitro or pre-clinical model, and assessment of potential toxicity.

In some embodiments of the invention, bi-specific or multi-specific antibodies are provided, which may have any of the configurations discussed herein. Various formats of bi-specific antibodies are within the ambit of the invention, including without limitation single chain polypeptides, two chain polypeptides, three chain polypeptides, four chain polypeptides, and multiples thereof.

Antigen binding domain (ABD). As used herein, the term ABD refers to a combination of variable heavy (VH and variable light (VL) polypeptides to associate to form a variable region domain. An ABD is the minimum antibody fragment that contains a complete antigen-recognition and binding site. This region consists of heavy- and one light-chain variable domain in tight, non-covalent association, as a single polypeptide or as a dimer. It is in this configuration that the three CDRS of each variable domain interact to define an antigen-binding site on the surface of the domain. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a B-sheet configuration, connected by three CDRs, which form loops connecting, and in some cases forming part of, the B-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

An antibody or ABR “which binds” an antigen of interest, is one that binds the antigen with sufficient affinity such that the antibody or binding molecule is useful as a diagnostic and/or therapeutic agent in targeting the antigen, and does not significantly cross-react with other proteins. In such embodiments, the extent of binding of the antibody or other binding molecule to a non-targeted antigen will usually be no more than 10% as determined by fluorescence activated cell sorting (FACS) analysis or radioimmunoprecipitation (RIA).

Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VH) domain. The two domains conventionally associate to form a specific binding region, although as well be discussed here, a variety of non-natural configurations of antibodies are known and used in the art.

A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or enzymatic activity. A functional antibody or other binding molecule may also block ligand activation of a receptor or act as an agonist or antagonist. The capability of an antibody or other binding molecule to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multi-specific antibodies (e.g., bi-specific antibodies), heavy chain only antibodies, three chain antibodies, single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. The immunoglobulins can be derived from any species. In one aspect, the immunoglobulin is of largely human origin.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Variable regions of interest include 3 CDR sequences, which may be obtained from available antibodies with the desired specificity, or may be obtained from antibodies developed for this purpose. One of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139., each of which is herein specifically incorporated by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or lgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis (ADCP); and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called κ and λ, based on the amino acid sequences of their constant domains.

A “functional Fc region” possesses an “effector function” of a native-sequence Fc region. Exemplary effector functions include C1q binding; CDC; Fc-receptor binding; ADCC; ADCP; down-regulation of cell-surface receptors (e.g., B-cell receptor), etc. Such effector functions generally require the Fc region to be interact with a receptor, e.g. the FcγRI; FcγRIIA; FcγRIIB1; FcγRIIB2; FcγRIIIA; FcγRIIIB receptors, and the low affinity FcRn receptor; and can be assessed using various assays as disclosed, for example, in definitions herein. A “dead” Fc is one that has been mutagenized to retain activity with respect to, for example, prolonging serum half-life, but which does not activate a high affinity Fc receptor. An Fc may also have decreased binding to complement.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563). Two amino acid substitutions in the complement Clq binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). Other Fc variants are possible, including without limitation one in which a region capable of forming a disulfide bond is deleted, or in which certain amino acid residues are eliminated at the N-terminal end of a native Fc form or a methionine residue is added thereto. Thus, in one embodiment of the invention, one or more Fc portions of the scFc molecule can comprise one or more mutations in the hinge region to eliminate disulfide bonding. In yet another embodiment, the hinge region of an Fc can be removed entirely. In still another embodiment, the molecule can comprise an Fc variant.

Further, an Fc variant can be constructed to remove or substantially reduce effector functions by substituting, deleting or adding amino acid residues to effect complement binding or Fc receptor binding. For example, and not limitation, a deletion may occur in a complement-binding site, such as a C1q-binding site. Techniques of preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The Fc may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms, e.g. Pichia pastoris, and mammalians cell line, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al, BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See e.g., WO 07/117505). The presence of particular glycosylation moieties has been shown to alter the function of Immunoglobulins. For example, the removal of sugar chains from an Fc molecule results in a sharp decrease in binding affinity to the Clq part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See e.g., Kaneko, et al, Science 313:760 (2006)), whereas removal of fucose from the IgG leads to enhanced ADCC activity (See e.g., Shoj-Hosaka, et al, J. Biochem., 140:777 (2006)).

In alternative embodiments, antibodies of the invention may have an Fc sequence with enhanced effector functions, e.g. by increasing their binding capacities to FcγRIIIA and increasing ADCC activity. For example, fucose attached to the N-linked glycan at Asn-297 of Fc sterically hinders the interaction of Fc with FcγRIIIA, and removal of fucose by glyco-engineering can increase the binding to FcγRIIIA, which translates into >50-fold higher ADCC activity compared with wild type IgG1 controls. Protein engineering, through amino acid mutations in the Fc portion of IgG1, has generated multiple variants that increase the affinity of Fc binding to FcγRIIIA. Notably, the triple alanine mutant S298A/E333A/K334A displays 2-fold increase binding to FcγRIIIA and ADCC function. S239D/1332E (2×) and S239D/1332E/A330L (3×) variants have a significant increase in binding affinity to FcγRIIIA and augmentation of ADCC capacity in vitro and in vivo. Other Fc variants identified by yeast display also showed the improved binding to FcγRIIIA and enhanced tumor cell killing in mouse xenograft models. See, for example Liu et al. (2014) JBC 289(6):3571-90, herein specifically incorporated by reference.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

“Fv” is the minimum antibody fragment, which contains a complete antigen-recognition and antigen-binding site. The CD3 binding antibodies of the invention comprise a dimer of one heavy chain and one light chain variable domain in tight, non-covalent association; however additional antibodies, e.g. for use in a multi-specific configuration, may comprise a VH in the absence of a VL sequence. Even a single variable domain (or half of an Fv comprising only three hypervariable regions specific for an antigen) has the ability to recognize and bind antigen, although the affinity may be lower than that of two domain binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

“Humanized” forms of non-human (e.g., rodent) antibodies, including single chain antibodies, are chimeric antibodies (including single chain antibodies) that contain minimal sequence derived from non-human immunoglobulin. See, for example, Jones et al, (1986) Nature 321:522-525; Chothia et al (1989) Nature 342:877; Riechmann et al (1992) J. Mol. Biol. 224, 487-499; Foote and Winter, (1992) J. Mol. Biol. 224:487-499; Presta et al (1993) J. Immunol. 151, 2623-2632; Werther et al (1996) J. Immunol. Methods 157:4986-4995; and Presta et al (2001) Thromb. Haemost. 85:379-389. For further details, see U.S. Pat. Nos. 5,225,539; 6,548,640; 6,982,321; 5,585,089; 5,693,761; 6,407,213; Jones et al (1986) Nature, 321:522-525; and Riechmann et al (1988) Nature 332:323-329.

The antibodies of the invention are typically used in multi-specific configurations, which include without limitation bi-specific antibodies, trifunctional antibodies, etc. A large variety of methods and protein configurations are known and use in bi-specific monoclonal antibodies (BsMAB), tri-specific antibodies, etc. For example, see Suursa et al. (2019) Pharmacology & Therapeutics 201:103-119; and Labrijn et al. (2019) Nature Reviews Drug Discovery 18:585-608. Recombinant proteins can force the correct association of heavy-light chains and the heavy chains by multiple means. Examples are the knob-in-holes approach where one heavy chain is engineered with a knob consisting of relatively large amino acids and the other heavy chain is engineered with a hole consisting of relatively small amino acids (A. M. Merchant et al., 1998) and similar molecules. Other examples are the constructs with their fragments connected by peptide chains, such as bi-specific T cell engagers (BiTE) molecules, thereby circumventing random association of the chains (Mack, Riethmuller, & Kufer, 1995).

Many formats of bi-specific antibodies are known and used in the art. First-generation BsMAbs consisted of two heavy and two light chains, one each from two different antibodies. The two Fab regions are directed against two antigens. The Fc region is made up from the two heavy chains and forms the third binding site with the Fc receptor on immune cells (see for example Lindhofer et al., The Journal of Immunology, Vol 155, p 219-225, 1995). The antibodies may be from the same or different species. For example, cell lines expressing rat and mouse antibodies secrete functional bi-specific Ab because of preferential species-restricted heavy and light chain pairing. In other embodiments the Fc regions are designed to only fit together in specific ways.

Other types of bi-specific antibodies include chemically linked Fabs, consisting only of the Fab regions. Two chemically linked Fab or Fab2 fragments form an artificial antibody that binds to two different antigens, making it a type of bi-specific antibody. Antigen-binding fragments (Fab or Fab2) of two different monoclonal antibodies are produced and linked by chemical means like a thioether (see Glennie, M J et al., Journal of immunology 139, p 2367-75, 1987; Peter Borchmann et al., Blood, Vol. 100, No. 9, p 3101-3107, 2002).

Methods for the production of multivalent artificial antibodies have been developed by recombinantly fusing variable domains of two antibodies. A single-chain variable fragment (scFv) is a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. Bi-specific single-chain variable fragments (di-scFvs, bi-scFvs) can be engineered by linking two scFvs with different specificities. A single peptide chain with two VH and two VL regions is produced, yielding bivalent scFvs.

Bi-specific tandem scFvs are also known as bi-specific T-cell engagers (BiTEs). Bi-specific scFvs can be created with linker peptides that are too short for the two variable regions to fold together (about five amino acids), forcing scFvs to dimerize. This type is known as diabodies (Adams et al., British journal of cancer 77, p 1405-12, 1998). The Dual-Affinity Re-Targeting (DART) platform technology (Macrogenics, Rockville, Md.). This fusion protein technology uses two single-chain variable fragments (scFvs) of different antibodies on a single peptide chain of about 55 kilodaltons. SCORPION Therapeutics (Emergent Biosolutions, Inc., Seattle, Wash.) combines two antigen-binding domains in a single chain protein. One binding domain is on the C-terminus and a second binding domain on the N-terminus of an effector domain, based on immunoglobulin Fc regions.

Tetravalent and bi-specific antibody-like proteins also include DVD-Igs which are engineered from two monoclonal antibodies (Wu, C. et al., Nature Biotechnology, 25, p 1290-1297, 2007). To construct the DVD-Ig molecule, the V domains of the two mAbs are fused in tandem by a short linker (TVAAP) with the variable domain of the first antibody light (VL) chain at the N terminus, followed by the other antibodies VL and Ck to form the DVD-Ig protein light chain. Similarly, the variable regions of the heavy (VH) chain of the two mAbs are fused in tandem by a short linker (ASTKGP) with the first antibody at the N terminus, followed by the other antibody and the heavy chain constant domains to form the DVD-Ig protein heavy chain (VH1/VL1). All light chain and heavy chain constant domains are preserved in the DVD-Ig design, as they are critical for the formation of a disulfide-linked full IgG-like molecule. Cotransfection of mammalian cells with expression vectors encoding the DVD-Ig light chain and heavy chain leads to the secretion of a single species of an IgG-like molecule with molecular weight of approximately 200 kDa. This molecule has now four binding sites, 2 from each mAb.

Heavy chain antibodies constitute about one fourth of the IgG antibodies produced by the camelids, e.g. camels and llamas (Hamers-Casterman C., et al. Nature. 363, 446-448 (1993)). These antibodies are formed by two heavy chains but are devoid of light chains. As a consequence, the variable antigen binding part is referred to as the VHH domain and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

### Conditions for Treatment.

Conditions for which the antibodies described herein find use in treatment include a number of inflammatory conditions where inhibition of activated B or T cells is of interest. Inflammation contributes to pathogenesis of inflammatory diseases, or diseases associated with inflammation, such as autoimmune diseases including rheumatoid arthritis (RA), systemic lupus erythematosus (SLE), multiple sclerosis (MS), and autoimmune hepatitis; degenerative diseases with an inflammatory component such as osteoarthritis (OA), Alzheimer's disease (AD), and macular degeneration; chronic infections such as HIV; metabolic diseases with an inflammatory component such as type II diabetes, metabolic syndrome, and atherosclerosis; and malignant diseases including cancers. Treatment may comprise administering an effective dose of a bi-specific antibody to reduce activated immune cell activity.

Inflammation associated with B cell activation is of interest, which conditions include, without limitation, systemic lupus erythematosus (SLE), myasthenia gravis, rheumatoid arthritis, IgG4 related disease; etc. B cell functions including autoantibody production, antigen presentation, T cell help and cytokine production all contribute to the pathogenesis of autoimmune diseases. There are multiple mechanisms by which autoantibodies contribute to the pathogenesis of autoimmune disease. First, immune complexes can form or deposit in tissues where they can activate complement and induce CDC to cause tissue damage. RA is associated with the production of rheumatoid factor, autoantibodies that bind the Fc region of IgG, and anti-citrullinated protein antibodies (ACPAs). In RA, both rheumatoid factor containing and ACPA-containing immune complexes activate complement pathways in joints, leading to the production of C5a and the generation of the membrane attack complex (MAC), which both contribute to joint damage; IgM rheumatoid factor can also increase complement activation mediated by ACPA-containing immune complexes. Autoantibodies can promote tissue damage via ADCC by co-engagement of antigens on the target tissue and Fc receptors (FcRs) on macrophages, neutrophils, natural killer (NK) cells and other effector cell types. Autoantibody containing immune complexes can also activate immune cells through dual engagement of FcRs and Toll-like receptors (TLRs) (on macrophages and dendritic cells) or dual engagement of the B cell receptor (BCR) and TLRs. Immune complexes facilitate antigen loading onto dendritic cells via immune complexes, enabling these cells to efficiently activate T cells.

Pathological interactions between B cells and T cells can also contribute to autoimmunity. B cells can function as professional APCs through constitutive expression of MHC class II molecules. B cells can also interact with and activate CD4+ T cells via MHC class II mediated antigen presentation, and CD4+ T cells, in turn, provide help to cognate B cells. During an immune response, naive CD4+ T cells are primed by antigen presenting dendritic cells and subsequently differentiate into T helper cell subsets including T follicular helper (TFH) cells. In the germinal center, TFH cells interact with cognate B cells to promote isotype switching and somatic hypermutation, as well as B cell differentiation into memory B cells and plasma cells.

Dysregulated antigen presentation is implicated in the pathogenesis of autoimmunity. Pathological T cell-B cell interactions are also implicated by the presence of ectopic lymphoid structures in inflamed tissues, as well as by evidence of autoantibody affinity maturation. In addition to B cell-TFH cell interactions, interactions between B cells and other cells can be dysregulated in autoimmunity. Targeting these interactions can be effective for preventing the development and progression of autoimmune diseases.

In addition to autoantibody production and antigen presentation, B cells can regulate immune responses through the production of cytokines. B cells can produce pro-inflammatory cytokines such as IFNγ, TNFα, IL2, IL4, IL-6, and IL21 as well as anti-inflammatory cytokines such as IL-10. Multiple sclerosis, in particular, involves perturbations in cytokine production by B cells, but B cell-mediated cytokine secretion is also perturbed in other autoimmune diseases such as SLE and RA. The production of IL-6 and IFNγ by B cells is required for spontaneous germinal center formation and TFH cell differentiation in SLE, MS, etc. Therapeutically targeting B cells can prevent loss of tolerance in both B cells and T cells as well as reduce inflammatory responses that can promote autoimmunity.

In contrast to pro-inflammatory B cell responses, regulatory B (Breg) cells are characterized by the production of anti-inflammatory cytokines such as IL-10, TGFβ and IL-35. IL-10-producing Breg cells (so-called B10 cells) have important protective functions against the development of autoimmunity, including in mouse models of autoimmunity and in multiple sclerosis, SLE and RA. TGFβ is produced by some Breg cells and can also modulate T cell activity. Additionally, a population of IL-35-expressing Breg cells can suppress autoimmunity.

B cell inhibitory checkpoints serve to both inhibit activation of autoreactive B cells and dampen overstimulated responses. For non-polyvalent antigens, two signals are required for the activation of B cells: the engagement of the BCR and a co-stimulatory signal. Antigen binding to the BCR defines the specificity of the B cell response, whereas co-stimulatory functions are important for overcoming inhibitory checkpoints. Once B cells migrate to the periphery, additional inhibitory checkpoint pathways ensure that residual autoreactive B cells are eliminated or silenced, whereas co-stimulatory signals ensure that only B cells with non-autoreactive BCRs are activated. Many of these stimulatory and inhibitory checkpoint pathways function indirectly to control the survival, proliferation and activation of B cells.

B cell activation by antigens is dependent on non-covalent interactions between the BCR and the invariant Igα and Igβ chains (also known as CD79A and CD79B). These chains contain immunoreceptor tyrosine-based activation motifs (ITAMs) that, upon antigen engagement of the BCR, are phosphorylated by the tyrosine kinase LYN60. Downstream activation signals are then amplified and relayed by the SYK and BTK kinases. Thus, BCR activation is mediated by activating co-receptors and their associated kinases, which requires overcoming constitutive inhibitory signals from inhibitory co-receptors.

Cognate T cells promote B cell and plasma cell differentiation by providing co-stimulatory signals in the form of CD40 ligand (CD40L; also known as CD154). These cognate T cells are reciprocally activated by engagement of CD40L with CD40 on the surface of B cells. Without co-stimulation via CD40 or other receptors, BCR triggering can lead to apoptosis of the B cell instead of B cell activation. As an alternative to CD40-mediated co-stimulation of BCRs, B cells can be activated independently of T cells via dual stimulation of the BCR and TLRs. Engagement of BCRs by bacterial or viral structures in an antigen-specific manner and simultaneous engagement of TLRs in a PAMP-specific manner leads to activation of B cells. Dual stimulation of TLRs and BCRs probably contributes to autoantibody production in, and to the pathogenesis of SLE, myositis, Sjögren syndrome and other autoimmune rheumatic diseases in which autoreactive B cells and autoantibodies target proteins or molecular complexes containing DNA or RNA.

B cell activation can be facilitated by the activation of the co-receptor CD19. CD19 is an immunoglobulin superfamily glycoprotein associated with the BCR and is expressed on B cells from the preB cell stage through to the plasma cell differentiation stage. CD19 signals though the tyrosine kinases LYN and phosphoinositide 3-kinase (PI3K), which amplify signals from the BCR, decreasing the threshold for BCR activation. CD19 is an integral feature of the BCR signalling complex and is important for B cell activation. Both CD19 and the BCR are needed for B cell activation, whereas stimulation of either receptor alone leads to B cell apoptosis. CD19 is an immunomodulatory therapeutic target, in addition to CD20 and other B cell antigens, for the treatment of autoimmune diseases.

Many diseases have an underlying inflammatory component that contributes to disease initiation and/or progression. Thus, the spectrum of inflammatory diseases and diseases associated with inflammation is broad and includes autoimmune diseases such rheumatoid arthritis (RA), systemic lupus erythematosus (SLE), multiple sclerosis (MS), and autoimmune hepatitis; degenerative diseases such as osteoarthritis (OA), Alzheimer's disease (AD), and macular degeneration; chronic infections such as human immunodeficiency virus (HIV), chronic hepatitis C virus (HCV) infection, chronic hepatitis B virus (HBV), chronic cytomegalovirus (CMV) infection, tuberculosis (TB) infection, as well as other chronic viral and bacterial infections; metabolic diseases including type II diabetes, metabolic syndrome, non-alcoholic steatohepatitis (NASH), and alcoholic steatohepatitis; cardiovascular diseases such as atherosclerosis; cancers which can arise from and induce inflammation; as well as other diseases with an inflammatory component.

Rheumatoid Arthritis (RA) is a chronic syndrome characterized usually by symmetric inflammation of the peripheral joints, potentially resulting in progressive destruction of articular and periarticular structures, with or without generalized manifestations (Firestein (2003) Nature 423(6937):356-61; McInnes and Schett. (2011) N Engl J Med. 365(23):2205-19). The cause is unknown. A genetic predisposition has been identified, and, in some populations, localized to a pentapeptide in the HLA-DR beta1 locus of class II histocompatibility genes. Environmental factors may also play a role. For example, cigarette smoking places individuals possessing HLA-DR4 containing the “shared epitope” polymorphism at approximately 10-20 fold increased risk of developing RA. Cigarette smoking is thought to induce anti-citrullinated protein antibody (ACPA) responses, which are measured using the commercial cyclic-citrullinated peptide (CCP) assay (Klareskog et al. (2006) Arthritis Rheum. 54(1):38-46). In addition, periodontitis and infection with P. gingivalis might also play a role in the initiation of autoimmune responses that result in development of RA (Rutger and Persson. 2012, J Oral Microbiol. 4). Immunologic changes may be initiated by multiple factors. About 0.6% of all populations are affected, women two to three times more often than men. Onset may be at any age, most often between 25 and 50 yr.

Prominent immunologic abnormalities that may be important in pathogenesis include antibodies and immune complexes found in joint fluid cells and in vasculitis. Plasma cells produce antibodies that contribute to these complexes. Lymphocytes that infiltrate the synovial tissue are primarily T helper cells, which can produce pro-inflammatory cytokines. Macrophages and their cytokines (e.g., tumor necrosis factor, granulocyte-macrophage colony-stimulating factor) are also abundant in diseased synovium. Increased adhesion molecules contribute to inflammatory cell emigration and retention in the synovial tissue. Increased macrophage-derived lining cells are prominent along with some lymphocytes and vascular changes in early disease.

In chronically affected joints, the normally delicate synovium develops many villous folds and thickens because of increased numbers and size of synovial lining cells and colonization by lymphocytes and plasma cells. The lining cells produce various materials, including collagenase and stromelysin, which can contribute to cartilage destruction; interleukin-1, which stimulates lymphocyte proliferation; and prostaglandins. The infiltrating cells, initially perivenular but later forming lymphoid follicles with germinal centers, synthesize interleukin-2, other cytokines, RF, and other immunoglobulins. Fibrin deposition, fibrosis, and necrosis also are present. Hyperplastic synovial tissue (pannus) may erode cartilage, subchondral bone, articular capsule, and ligaments. PMNs are not prominent in the synovium but often predominate in the synovial fluid.

Onset is usually insidious, with progressive joint involvement, but may be abrupt, with simultaneous inflammation in multiple joints. Tenderness in nearly all inflamed joints is the most sensitive physical finding. Synovial thickening, the most specific physical finding, eventually occurs in most involved joints. Symmetric involvement of small hand joints (especially proximal interphalangeal and metacarpophalangeal), foot joints (metatarsophalangeal), wrists, elbows, and ankles is typical, but initial manifestations may occur in any joint. RA is characterized by the development of focal bone erosions through degradation and remodeling of bone at the joint margins and in subchondral bone of patients with RA. A hallmark of a subset of RA is the development of autoantibodies, including rheumatoid factors (RF) and anti-citrullinated protein antibodies (ACPA). RF, antibodies to human Fcγ, are present in about 70% of patients with RA. However, RF, often in low titers, occurs in patients with other diseases, including other connective tissue diseases such as systemic lupus erythematous, granulomatous diseases, chronic infections such as viral hepatitis, subacute bacterial endocarditis, and tuberculosis, and cancers. Low RF titers can also occur in a small percentage of the general population, and more commonly in the elderly. Another disease indicator is the presence of ACPA, which are measured using the clinical anti-CCP (cyclic citrullinated peptide) antibody test. Anti-CCP antibodies are approximately 60% sensitive and 95% specific for the diagnosis of RA, and like RF, predict a worse prognosis.

Systemic lupus erythematosus (SLE) is a systemic autoimmune disease characterized by malar rashes, oral ulcers, photosensitivity, serositis, seizures, low white blood cell counts, low platelet counts, seizures, a positive anti-nuclear antibody (ANA) test, and other positive autoantibodies. SLE is an autoimmune disease characterized by polyclonal B cell activation, which results in a variety of anti-protein and non-protein autoantibodies that result in immune complexes and inflammation which contributes to tissue damage (see, e.g., Kotzin et al., 1996, Cell 85:303-06 for a review of the disease). SLE has a variable course characterized by exacerbations and remissions and is difficult to study. For example, some patients may demonstrate predominantly skin rash and joint pain, show spontaneous remissions, and require little medication. The other end of the spectrum includes patients who demonstrate severe and progressive kidney involvement (glomerulonephritis and cerebritis) that requires therapy with high doses of steroids and cytotoxic drugs such as cyclophosphamide. Hydroxychloroquine slows SLE progression, and is a mainstay therapeutic for the management of SLE.

Multiple sclerosis (MS) is a debilitating, inflammatory, neurological illness characterized by demyelination of the central nervous system. The disease primarily affects young adults with a higher incidence in females. Symptoms of the disease include fatigue, numbness, tremor, tingling, dysesthesias, visual disturbances, dizziness, cognitive impairment, urological dysfunction, decreased mobility, and depression. Four types classify the clinical patterns of the disease: relapsing-remitting, secondary progressive, primary-progressive and progressive-relapsing (S. L. Hauser and D. E. Goodkin, Multiple Sclerosis and Other Demyelinating Diseases in Harrison's Principles of Internal Medicine 14th Edition, vol. 2, Mc Graw-Hill, 1998, pp. 2409-19).

Inflammatory bowel diseases, include Crohn's disease and ulcerative colitis, involve autoimmune attack of the bowel. These diseases cause chronic diarrhea, frequently bloody, as well as symptoms of colonic dysfunction.

Systemic sclerosis (SSc, or scleroderma) is an autoimmune disease characterized by fibrosis of the skin and internal organs and widespread vasculopathy. Patients with SSc are classified according to the extent of cutaneous sclerosis: patients with limited SSc have skin thickening of the face, neck, and distal extremities, while those with diffuse SSc have involvement of the trunk, abdomen, and proximal extremities as well. Internal organ involvement tends to occur earlier in the course of disease in patients with diffuse compared with limited disease (Laing et al. (1997) Arthritis. Rheum. 40:734-42). The majority of patients with diffuse SSc who develop severe internal organ involvement will do so within the first three years after diagnosis at the same time the skin becomes progressively fibrotic (Steen and Medsger (2000) Arthritis Rheum. 43:2437-44.). Common manifestations of diffuse SSc that are responsible for substantial morbidity and mortality include interstitial lung disease (ILD), Raynaud's phenomenon and digital ulcerations, pulmonary arterial hypertension (PAH) (Trad et al. (2006) Arthritis. Rheum. 54:184-91.), musculoskeletal symptoms, and heart and kidney involvement (Ostojic and Damjanov (2006) Clin. Rheumatol. 25:453-7). Current therapies focus on treating specific symptoms, but disease-modifying agents targeting the underlying pathogenesis are lacking.

Autoimmune hepatitis is a disease in which the body's immune system attacks liver cells. This immune response causes inflammation of the liver, also called hepatitis. Researchers think a genetic factor may make some people more susceptible to autoimmune diseases. About 70 percent of those with autoimmune hepatitis are female. The disease is usually quite serious and, if not treated, gets worse over time. Autoimmune hepatitis is typically chronic, meaning it can last for years, and can lead to cirrhosis—scarring and hardening—of the liver. Eventually, liver failure can result.

The presence of inflammation can be detected by a variety of approaches, including clinical history, physical examination, laboratory testing, histologic analysis of tissue, analysis of biomarkers, and imaging. Clinical features and physical exam markers of inflammation include swelling, effusions, edema, redness, warmth, pain, or associated pathologically with the influx of inflammatory cells or production of inflammatory mediators. Laboratory testing and/or histologic markers are abnormal when increased numbers of inflammatory cells are demonstrated. Markers of inflammation can include a molecular marker(s), and examples of a molecular marker(s) include C-reactive protein, a cytokine, an antibody, a DNA sequence, an RNA sequence, a cartilage marker, a metabolic marker, a bone marker, or combinations thereof. Imaging can reveal findings including enhancement of tissues, edema and swelling of tissues, and other findings indicative of inflammation. Examples of imaging markers of inflammation can include imaging markers measured using magnetic resonance imaging, ultrasound, computed tomography, angiography, and combinations thereof.

The presence of low-grade inflammation is characterized by an elevation(s) in the local or systemic concentrations of cytokines such as TNF-.alpha., IL-6, and c-reactive protein (CRP), and occurs in adiposity, osteoarthritis, Alzheimer's disease, type II diabetes, metabolic syndrome, coronary artery disease, nonalcoholic fatty liver disease (NAFLD), non-alcoholic steatohepatitis, and many chronic and degenerative diseases. Low-grade inflammation is manifest by inflammation present at a level below the “high-grade” inflammation detected in active autoimmune diseases (such as active rheumatoid arthritis, psoriasis, Crohn's disease, systemic lupus erythematous, autoimmune hepatitis, and other autoimmune states) and in certain viral and bacterial infections during which humans experience clinical symptoms (such as influenza virus infection, Staphylococcus aureus infection, and other infections).

The reduction or amelioration of inflammation is indicated by dissipation of inflammation, a reduction in number of inflammatory cells or in levels of inflammatory mediators as evidenced by symptomatic relief (including but not limited to pain relief), radiographic changes, biochemical changes, pathologic/histologic changes, decreased progression of such markers of inflammation, decreased development of findings indicative of tissue or organ damage, decreased development of symptoms or signs of disease, or decreased development of disease.

In some embodiments the methods of the invention comprise the step of identifying individuals “at-risk” for development of, or in the “early-stages” of, an inflammatory disease. “At risk” for development of an inflammatory disease includes: (1) individuals whom are at increased risk for development of an inflammatory disease, and (2) individuals exhibiting a “pre-clinical” disease state, but do not meet the diagnostic criteria for the inflammatory disease (and thus are not formally considered to have the inflammatory disease).

Individuals “at increased risk” for development (also termed “at-risk” for development) of an inflammatory disease are individuals with a higher likelihood of developing an inflammatory disease or disease associated with inflammation compared to the general population. Such individuals can be identified based on their exhibiting or possessing one or more of the following: a family history of inflammatory disease; the presence of certain genetic variants (genes) or combinations of genetic variants which predispose the individual to such an inflammatory disease; the presence of physical findings, laboratory test results, imaging findings, marker test results (also termed “biomarker” test results) associated with development of the inflammatory disease, or marker test results associated with development of a metabolic disease; the presence of clinical signs related to the inflammatory disease; the presence of certain symptoms related to the inflammatory disease (although the individual is frequently asymptomatic); the presence of markers (also termed “biomarkers”) of inflammation; and other findings that indicate an individual has an increased likelihood over the course of their lifetime to develop an inflammatory disease or disease associated with inflammation. Most individuals at increased risk for development of an inflammatory disease or disease associated with inflammation are asymptomatic, and are not experiencing any symptoms related to the disease that they are at an increased risk for developing.

Included, without limitation, in the group of individuals at increased risk of developing an inflammatory disease or a disease associated with inflammation, are individuals exhibiting “a pre-clinical disease state”. The pre-disease state may be diagnosed based on developing symptoms, physical findings, laboratory test results, imaging results, and other findings that result in the individual meeting the diagnostic criteria for the inflammatory disease, and thus being formally diagnosed. Individuals with “pre-clinical disease” exhibit findings that suggest that the individual is in the process of developing the inflammatory disease, but do not exhibit findings, including the symptoms, clinical findings, laboratory findings, and/or imaging findings, etc. that are necessary to meet the diagnostic criteria for a formal diagnosis of the inflammatory disease. In some embodiments, individuals exhibiting a pre-clinical disease state possess a genetic variant or a combination of genetic variants that place them at increased risk for development of disease as compared to individuals who do not possess that genetic variant or that combination of genetic variants. In some embodiments, these individuals have laboratory results, or physical findings, or symptoms, or imaging findings that place them at increased risk for development of an inflammatory disease. In some embodiments, individuals with preclinical disease states are asymptomatic. In some embodiments, individuals with pre-clinical disease states exhibit increased or decreased levels of the expression of certain genes, certain proteins, inflammatory markers, metabolic markers, and other markers.

In some embodiments, individuals at increased risk for an inflammatory disease exhibit increased markers of inflammation (also termed “inflammatory markers” or “inflammatory biomarkers”). Examples of molecular markers of inflammation include c-reactive protein (CRP), high-sensitivity CRP (hs-CRP) (or regular CRP), erythrocyte sedimentation rate (ESR), serum amyloid A, serum amyloid P, fibrinogen, cytokines in blood or other biological fluids, a cytokine, an antibody (such as an autoantibody, or an anti-microbial antibody), a DNA sequence, a RNA sequence (for example, mRNA encoding one or more cytokines or other immune molecules), other markers of inflammation, or combinations thereof. The method can include determining the presence of inflammation prior to treatment, for example by detection and analysis of one or more markers of inflammation, where an individual in an early stage of disease showing signs of inflammation is selected for treatment with a formulation of the invention. In some embodiments the treatment ameliorates, diminishes, actively treats, reverses or prevents tissue injury. In some embodiments the inflammatory disease is an autoimmune disease, for example RA, multiple sclerosis, systemic lupus erythematosus, Sjogren's Syndrome, etc. In some embodiments, the marker of inflammation (or inflammatory marker) is a metabolic marker (also termed herein as a “metabolic disease marker”). In some embodiments the disease comprises an inflammatory component contributing to a metabolic disease, for example metabolic syndrome, type II diabetes, insulin resistance, atherosclerosis, etc. In some embodiments the disease is a degenerative disease such as OA, Alzheimer's disease, or macular degeneration.

In some embodiments the marker of inflammation is an abnormal metabolic marker (also termed herein as a “metabolic disease marker”), and the abnormal metabolic marker is selected from the group consisting of a blood pressure of about 140/90 mmHg or more, plasma triglyceride levels of about 1.7 mmol/L or more, high-density lipoprotein cholesterol (HDL-C) levels of about 0.9 mmol/L or less for males and about 1.0 mmol/L or less for females, a microalbuminuria:urinary albumin excretion ratio of about 20.mu.g/min or more, or an albumin:creatinine ratio of about 30 mg/g or more, a fasting blood glucose greater than about 100 mg/dL, 2 hour post-prandial blood glucose greater than about 200, or a hemoglobin A1c greater than about 6.5 mg/dL. Patients with abnormal metabolic markers are at increased risk for developing an inflammatory disease or disease associated with inflammation.

### Methods of Use

Methods are provided for treating or reducing disease, including without limitation infection, autoimmune disease or inflammatory disease in a regimen comprising contacting the targeted cells with an antigen-binding composition of the invention, particularly where the antigen-binding composition is a multi-specific antibody suitable for the condition being treated. Such methods include administering to a subject in need of treatment a therapeutically effective amount or an effective dose of the agents of the invention, including without limitation combinations of the reagent with a additional drugs, e.g. steroids, DMARDs, and the like.

The methods of the invention can be used for prophylactic as well as therapeutic purposes. As used herein, in one embodiment the term “treating” refers to prophylactic or preventative use of the intervention in individuals with increased risk for or with early-stage inflammatory disease. In such individuals, treatment prevents development of symptoms or signs of disease, prevents development of disease, and/or reverses signs or symptoms of disease. In another embodiment, the term “treating” refers to treating individuals with established disease to reduce symptoms or signs of disease, to prevent disease progression, and/or to reverse symptoms of signs of disease.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration. Dosage unit forms generally contain between from about 1 mg to about 500 mg of an active ingredient.

In some embodiments, the therapeutic dosage the agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Compositions for the treatment of disease can be administered by parenteral, topical, intravenous, intratumoral, oral, subcutaneous, intraarterial, intracranial, intraperitoneal, intranasal or intramuscular means. A typical route of administration is intravenous or intralesional, although other routes can be equally effective.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the proteins described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents and formulations thereof, of the invention and instructions for use. The kit can further contain a least one additional reagent, e.g. a chemotherapeutic drug, etc. Kits typically include a label indicating the intended use of the contents of the kit. The term label includes any writing, or recorded material supplied on or with the kit, or which otherwise accompanies the kit.

The compositions can be administered for therapeutic treatment. Compositions are administered to a patient in an amount sufficient to substantially ablate targeted cells, as described above. An amount adequate to accomplish this is defined as a “therapeutically effective dose.”, which may provide for an improvement in overall survival rates. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

In one embodiment, this invention provides for the treatment of individuals with established inflammatory disease. The inflammatory disease is diagnosed based on an individual exhibiting symptoms, signs, clinical features, laboratory test results, imaging test results, marker results, and other findings that enable a physician to formally diagnose that individual with the inflammatory disease. In some embodiment, established inflammatory disease is an inflammatory disease for which an individual has had a formal diagnosis of the disease made by a physician for longer than 6 months. In established inflammatory disease, the signs or symptoms of disease may be more severe. In established inflammatory disease, the disease process may cause tissue or organ damage.

In some embodiments the methods of the invention comprise the step of identifying and treating individuals at increased risk for development of an inflammatory disease or disease associated with inflammation. These individuals at increased risk for development of an inflammatory disease can have risk factors for disease and/or be in a “pre-clinical” state as described herein, and are sometimes asymptomatic.

The determination of “early-stage disease” in an individual can comprise analyzing the individual for the presence of at least one marker indicative of the presence of early disease. In some embodiments the method comprises analyzing an individual for the presence of one, two, three, four, or more markers that are predictive for an individual being at increased risk for developing or in the early-stages of an inflammatory disease or disease associated with inflammation. In some embodiments at least one of the marker(s) is an imaging marker, including without limitation: arthroscopy, radiographic imaging, ultrasound imaging, magnetic resonance imaging (MRI), computed tomography (CT), etc. In some embodiments at least one of the marker(s) is a molecular marker of inflammation, where a biological sample is obtained from the individual and analyzed for the presence of a molecule, e.g. high-sensitivity C-reactive protein (or regular CRP), erythrocyte sedimentation rate, serum amyloid A, serum amyloid P, fibrinogen, a cytokine, antibody (autoantibody or anti-microbial antibody), cartilage component, protease, RNA molecule (for example, mRNA encoding one or more cytokines or other immune molecules), etc. and compared to a control or reference value, wherein altered level of the molecular marker is indicative of early disease. In some embodiments, early-stage disease is defined by the presence of symptoms for less than about 6 months. In some embodiments, early-stage disease is defined by being formally diagnosed with the inflammatory disease for less than about 6 months. In some embodiments, early-stage disease is associated with no symptoms. In some embodiments, early-stage disease is associated with mild symptoms. In some embodiments, early-stage disease is associated with intermittent symptoms, such as symptoms occurring only once every couple years, or symptoms occurring once every couple months, or symptoms occurring once every couple days, or symptoms occurring for only part of each day. Such individuals identified as having early-stage inflammatory disease can then be treated, advantageously, with a bi-specific antibody as described herein.

In certain embodiments, this invention is directed to the treatment of individuals with established inflammatory disease or disease associated with inflammation. The inflammatory disease is diagnosed based on an individual exhibiting symptoms, signs, clinical features, laboratory test results, imaging test results, biomarker results, and other findings that enable a physician to formally diagnose that individual with the inflammatory disease. In some embodiments, established inflammatory disease is an inflammatory disease for which an individual has had a formal diagnosis of the disease made by a physician for longer than 6 months. In established inflammatory disease, the signs or symptoms of disease may be more severe as compared to, for example, the symptoms for an individual diagnosed with early-stage inflammatory disease. In established inflammatory disease, the disease process may cause tissue or organ damage. As described herein, in certain embodiments, determination of inflammation in an individual with established disease can comprise analyzing the individual for the presence of at least one marker indicative of the presence of inflammation.

Determination of inflammation in an individual at risk for, with early-stage, or with established disease can comprise analyzing the individual for the presence of at least one marker indicative of the presence of inflammation or metabolic abnormalities. Markers of inflammation include molecular markers, metabolic markers, clinical markers and imaging markers. In some embodiments, the method comprises analyzing an individual for the presence of one, two, three, four, or more markers of inflammation that are diagnostic for inflammation, which can be systemic or localized inflammation. In some embodiments at least one of the marker(s) of inflammation is an imaging marker, including without limitation radiographic imaging, ultrasound imaging, magnetic resonance imaging (MRI), computed tomography (CT), etc. In some embodiments at least one of the marker(s) of inflammation is a molecular marker, where a biological sample is obtained from the individual and analyzed for the presence of a molecule, e.g. high-sensitivity CRP (or regular CRP), cytokine, serum amyloid A, serum amyloid P, fibrinogen, antibody (autoantibody or anti-microbial antibody), cartilage component, protease, RNA sequence (for example, mRNA encoding one or more cytokines or other immune molecules), etc. and compared to a control or reference value, wherein altered level of the molecular marker is indicative of inflammation. In some embodiments, the marker of inflammation is an abnormal clinical marker. Examples of abnormal clinical markers of inflammation include swelling on physical examination, tenderness on physical examination, and combinations thereof. In some embodiments the marker indicative of inflammation indicates the presence of local inflammation, i.e. inflammation present at the affected joint, in the central nervous system, or in another tissue, organ or site within the body. In some embodiments, the marker of inflammation is an abnormal metabolic marker, and the abnormal metabolic marker is selected from the group consisting of a blood pressure of about 140/90 mmHg or more, plasma triglyceride levels of about 1.7 mmol/L or more, high-density lipoprotein cholesterol (HDL-C) levels of about 0.9 mmol/L or less for males and about 1.0 mmol/L or less for females, a microalbuminuria:urinary albumin excretion ratio of about 20.mu.g/min or more, or an albumin:creatinine ratio of about 30 mg/g or more, a fasting blood glucose greater than about 100 mg/dL, 2 hour post-prandial blood glucose greater than about 200, or a hemoglobin A1c greater than about 6.5 mg/dL. The measurement or detection of an abnormal inflammatory or metabolic marker in an individual is predictive for that individual being at increased risk of developing, in the pre-clinical phase of, in the early-stages of, or having an established inflammatory disease or disease associated with inflammation.

Administration of a bi-specific antibody as described herein to an animal, human or other mammal includes administration via any route including but not limited to oral, intradermal, intramuscular, intraperitoneal, or intravenous.

In one embodiment, provided is a method of treating an inflammatory disease that includes administering to a patient an effective dose of a bi-specific antibody in combination with a statin, or in combination with one or more therapeutic compounds, including without limitation a cytokine; an antibody, e.g. Tysabri (natalizumab); fingolimod (Gilenya); copaxone, etc. The effective dose of each drug in a combination therapy may be lower than the effective dose of the same drug in a monotherapy. In some embodiments the combined therapies are administered concurrently. In some embodiments the two therapies are phased, for example where one compound is initially provided as a single agent, e.g. as maintenance, and where the second compound is administered during a relapse, for example at or following the initiation of a relapse, at the peak of relapse, etc.

### Producing Proteins

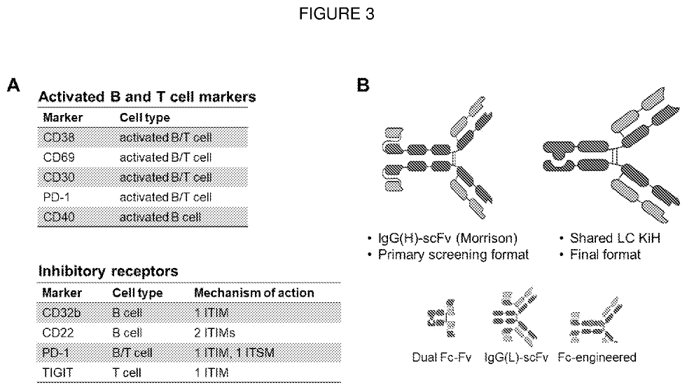

Antibody variable regions targeting activated B cell markers and inhibitor receptors of interest are provided in Table 2. These variable regions can be used to generate bi-specific antibodies of a variety of bi-specific and multi-specific antibody formats including the formats presented in FIG. 3, which provide schematic views of the IgG(H)-scFv Morrison format, the Shared LC KiH forma, the dual Fc-Fv format, the Ig(L)-scFv format, or the Fc-engineered bispcific format.

Although antibodies can be prepared by chemical synthesis, they are typically produced by methods of recombinant DNA technology, such as co-expression of all the chains making up the protein in a single recombinant host cell, or co-expression of a heavy chain polypeptide and an antibody, e.g. a human antibody. In addition, the antibody heavy and light chains can also be expressed using a single polycistronic expression vector. Purification of individual polypeptides is achieved using standard protein purification technologies such as affinity (protein A) chromatography, size exclusion chromatography and/or hydrophobic interaction chromatography. Bi-specifics are sufficiently different in size and hydrophobicity that purification can be performed using standard procedures.

The amount of antibody and heavy chain polypeptide produced in a single host cell can be minimized through engineering of constant regions of the antibody and the heavy chain such that homodimerization is favored over heterodimerization, e.g. by introducing self-complementary interactions (see e.g. WO 98/50431 for possibilities, such as “protuberance-into-cavity” strategies (see WO 96/27011)). It is therefore another aspect of the present invention to provide a method for producing a bi-specific in a recombinant host, the method including the step of: expressing in a recombinant host cell a nucleic acid sequences encoding at least two heavy chain polypeptides, wherein said heavy chain polypeptides differ in their constant regions sufficiently to reduce or prevent homodimer formation but increase bi-specific formation.

Where the protein comprises three chains, e.g. FlicAbs, they may be produced by co-expression of the three chains (2 heavy chains and one light chain) making up the molecule in a single recombinant host cell.

For recombinant production of the proteins herein, one or more nucleic acids encoding all chains, e.g. 2, 3 4, etc. are isolated and inserted into a replicable vector for further cloning (amplification of the DNA) or for expression. Many vectors are available. The vector components generally include, but are not limited to, one or more of the following: a signal sequence, an origin of replication, one or more marker genes, an enhancer element, a promoter, and a transcription termination sequence.

In a preferred embodiment, the host cell according to the method of the invention is capable of high-level expression of human immunoglobulin, i.e. at least 1 pg/cell/day, preferably at least 10 pg/cell/day and even more preferably at least 20 pg/cell/day or more without the need for amplification of the nucleic acid molecules encoding the single chains in said host cell.

### Pharmaceutical Composition

It is another aspect of the present invention to provide pharmaceutical compositions comprising one or more proteins of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

Therapeutic formulations of the proteins used in accordance with the present invention are prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Antibody formulations are disclosed, for example, in U.S. Patent Publication No. 20070065437, the entire disclosure is expressly incorporated by reference herein. Similar formulations can be used for the proteins of the present invention. The main components of such formulations are a pH buffering agent effective in the range of 3.0 to 6.2, a salt, a surfactant, and an effective amount of a multi-specific (bi-specific or other multi-specific) antibody.

The invention now being fully described, it will be apparent to one of ordinary skill in the art that various changes and modifications can be made without departing from the spirit or scope of the invention.

### EXAMPLES

### Example 1

Bi-specific antibodies were designed to bind to both a marker that defined activated B cell subsets (targeting molecule), and an inhibitory cell surface receptor, thereby creating proximity between both cell surface receptors.

Targeting molecules and inhibitory receptors were tested in preliminary screens, using the molecules shown in Table 1. For certain embodiments, the bi-specific and multi-specific sequences in Table 2 are utilized for one or more of the multi-specific (including bi-specific) binding domains. In other embodiments, other variable region(s) or other antibody-based binding sequences can be utilized for one or more of the multi-specific binding domains.

TABLE 2A

| Target | Chain | Sequence |
|---|---|---|
| PD-1 | VH | (SEQ ID NO: 1) |
| | | QVQLVESGGGVVQPGRSLRLDCKASGITFSNSGMHWVRQAPGKGLEWVAVIWYDGSKRY |
| | | YADSVKGRFTISRDNSKNTLFLQMNSLRAEDTAVYYCATNDDYWGQGTLVTVSS |
| | VL | (SEQ ID NO: 2) |
| | | EIVLTQSPATLSLSPGERATLSCRASQSVSSYLAWYQQKPGQAPRLLIYDASNRATGIPARF |
| | | SGSGSGTDFTLTISSLEPEDFAVYYCQQSSNWPRTFGQGTKVEIK |
| PD-L1 | VH | (SEQ ID NO: 3) |
| | | EVQLVESGGGLVQPGGSLRLSCAASGFTFSRYWMSWVRQAPGKGLEWVANIKQDGSEKY |
| | | YVDSVKGRFTISRDNAKNSLYLQMNSLRAEDTAVYYCAREGGWFGELAFDYWGQGTLVTV |
| | | SS |
| | VL | (SEQ ID NO: 4) |
| | | EIVLTQSPGTLSLSPGERATLSCRASQRVSSSYLAWYQQKPGQAPRLLIYDASSRATGIPDR |
| | | FSGSGSGTDFTLTISRLEPEDFAVYYCQQYGSLPWTFGQGTKVEIK |
| TIGIT | VH | (SEQ ID NO: 5) |
| | | QVQLQESGPGLVKPSETLSLTCAVSGYSITSDYAWNWIRQPPGKGLEWIGYISYSGSTSYN |
| | | PSLRSRVTISRDTSKNQFFLKLSSVTAADTAVYYCARRQVGLGFAYWGQGTLVTVSS |
| | VL | (SEQ ID NO: 6) |
| | | DIQMTQSPSSLSASVGDRVTITCKASQDVSTAVAWYQQKPGKAPKLLIYSASYRYTGVPSRF |
| | | SGSGSGTDFTFTISSLQPEDIATYYCQQHYSTPWTFGQGTKVEIK |
| CD22 | VH | (SEQ ID NO: 7) |
| | | QVQLVQSGAEVKKPGSSVKVSCKASGYTFTSYWLHWVRQAPGQGLEWIGYINPRNDYTEY |
| | | NQNFKDKATITADESTNTAYMELSSLRSEDTAFYFCARRDITTFYWGQGTTVTVSS |
| | VL | (SEQ ID NO: 8) |
| | | DIQLTQSPSSLSASVGDRVTMSCKSSQSVLYSANHKNYLAWYQQKPGKAPKLLIYWASTRE |
| | | SGVPSRFSGSGSGTDFTFTISSLQPEDIATYYCHQYLSSWTFGGGTKLEIK |
| CD69 | VH | (SEQ ID NO: 9) |
| | | QVQPGGSLRLSCAASGFTFSNFVMHWVRQAPGKGLEWVSSISGSSSSTYYADSVKGRFTI |
| | | SRDNSKNTLYLQMNSLRAEDTAVYYCARYYYASFDYWGQGTLVTVSS |
| | VL | (SEQ ID NO: 10) |
| | | DIELTQPPSVSVAPGQTARISCSGDSLGSKYVYWYQQKPGQAPVVVIYGDSKRPSGIPERFS |
| | | GSNSGNTATLTISGTQAEDEADYYCGSYTTGAKSHVFGGGTKLTVL |
| CD38 | VH | (SEQ ID NO: 11) |
| | | EVQLLESGGGLVQPGGSLRLSCAVSGFTFNSFAMSWVRQAPGKGLEWVSAISGSGGGTYY |
| | | ADSVKGRFTISRDNSKNTLYLQMNSLRAEDTAVYFCAKDKILWFGEPVFDYWGQGTLVTVS |
| | | S |
| | VL | (SEQ ID NO: 12) |
| | | EIVLTQSPATLSLSPGERATLSCRASQSVSSYLAWYQQKPGQAPRLLIYDASNRATGIPARF |
| | | SGSGSGTDFTLTISSLEPEDFAVYYCQQRSNWPPTFGQGTKVEIK |
| CD19 | VH | (SEQ ID NO: 13) |
| | | EVQLVESGGGLVKPGGSLKLSCAASGYTFTSYVMHWVRQAPGKGLEWIGYINPYNDGTKY |
| | | NEKFQGRVTISSDKSISTAYMELSSLRSEDTAMYYCARGTYYYGTRVFDYWGQGTLVTVSS |
| | VL | (SEQ ID NO: 14) |
| | | DIVMTQSPATLSLSPGERATLSCRSSKSLQNVNGNTYLYWFQQKPGQSPQLLIYRMSNLNS |
| | | GVPDRFSGSGSGTEFTLTISSLEPEDFAVYYCMQHLEYPITFGAGTKLEIK |
| CD79b | VH | (SEQ ID NO: 15) |
| | | QVQLVQSGAEVKKPGASVKVSCKASGYTFTSYWMNWVRQAPGQGLEWIGMIDPSDSETH |
| | | YNQKFKDRVTMTTDTSTSTAYMELRSLRSDDTAVYYCARAMGYWGQGTTVTVSS |
| | VL | (SEQ ID NO: 16) |
| | | DVVMTQSPLSLPVTLGQPASISCKSSQSLLDSDGKTYLNWFQQRPGQSPNRLIYLVSKLDS |
| | | GVPDRFSGSGSGTDFTLKISRVEAEDVGVYYCWQGTHFPLTFGGGTKLEIK |
| | | QVQLVESGPGLVKPSQSLSITCTVSGFSLSRYSVYWVRQPPGKGLEWLGMMWGGGSTDY |
| CD40 | VH | (SEQ ID NO: 17) |
| | | QVQLVESGPGLVKPSQSLSITCTVSGFSLSRYSVYWVRQPPGKGLEWLGMMWGGGSTDY |
| | | NSALKSRLTISKDTSKNQVFLKMNSLRAEDTAMYYCVRTDGDYWGQGTTVTVSS |
| | VL | (SEQ ID NO: 18) |
| | | DLQLTQSPLSLPVSLGDRASISCRSSQSLVNSNGNTYLHWYLQKPGQSPKLLIYKVSNRFSG |
| | | VPDRFSGSGSGTDFTLKISRVEAEDVGVYYCSQSTHVPWTFGGGTKLEIK |
| CD80 | VH | (SEQ ID NO: 19) |
| | | QVQLQESGPGLVKPSETLSLTCAVSGGSISGGYGWGWIRQPPGKGLEWIGSFYSSSGNTY |
| | | YNPSLKSQVTISTDTSKNQFSLKLNSMTAADTAVYYCVRDRLFSVVGMVYNNWFDVWGPG |
| | | VLVTVSS |
| | VL | (SEQ ID NO: 20) |
| | | ESALTQPPSVSGAPGQKVTISCTGSTSNIGGYDLHWYQQLPGTAPKLLIYDINKRPSGISDRF |
| | | SGSKSGTAASLAITGLQTEDEADYYCQSYDSSLNAQVFGGGTRLTVL |
| CD32b | VH | (SEQ ID NO: 21) |
| | | EVQLVESGGGLVQPGGSLRLSCAASGFTFSDAWMDWVRQAPGKGLEWVAEIRNKAKNHA |
| | | TYYAESVIGRFTISRDDAKNSLYLQMNSLRAEDTAVYYCGALGLDYWGQGTLVTVSS |
| | VL | (SEQ ID NO: 22) |
| | | DIQMTQSPSSLSASVGDRVTITCRASQEISGYLSWLQQKPGKAPRRLIYAASTLDSGVPSRF |
| | | SGSESGTEFTLTISSLQPEDFATYYCLQYFSYPLTFGGGTKVEIK |

TABLE 2B
CDR sequences from Table 2A

| Target | Chain | CDR1 | CDR2 | CDR3 |
|---|---|---|---|---|
| PD-1 | VH | (SEQ ID NO: 23) | (SEQ ID NO: 24) | (SEQ ID NO: 25) CATNDDYW |
| | | GITFSNSG | IWYDGSKR | |
| | VL | (SEQ ID NO: 26) | (SEQ ID NO: 27) | (SEQ ID NO: 28) CQQSSNWPRTF |
| | | QSVSSY | DAS | |
| PD-L1 | VH | (SEQ ID NO: 29) | (SEQ ID NO: 30) | (SEQ ID NO: 31) CAREGGWFGELAFDY W |
| | | GFTFSRYW | IKQDGSEK | |
| | VL | (SEQ ID NO: 32) | (SEQ ID NO: 33) | (SEQ ID NO: 34) CQQYGSLPWTF |
| | | QRVSSSY | DAS | |
| TIGIT | VH | (SEQ ID NO: 35) | (SEQ ID NO: 36) | (SEQ ID NO: 37) CARRQVGLGFAYW |
| | | GYSITSDYA | ISYSGST | |
| | VL | (SEQ ID NO: 38) | (SEQ ID NO: 39) | (SEQ ID NO: 40) CQQHYSTPWTF |
| | | QDVSTA | SAS | |
| CD22 | VH | (SEQ ID NO: 41) | (SEQ ID NO: 42) | (SEQ ID NO: 43) CARRDITTFYW |
| | | GYTFTSYW | INPRNDYT | |
| | VL | (SEQ ID NO: 44) | (SEQ ID NO: 45) | (SEQ ID NO: 46) CHQYLSSWTF |
| | | QSVLYSANHKNY | WAS | |
| CD69 | VH | (SEQ ID NO: 47) | (SEQ ID NO: 48) | (SEQ ID NO: 49) CARYYYASFDYW |
| | | GFTFSNFV | ISGSSSST | |
| | VL | (SEQ ID NO: 50) | (SEQ ID NO: 51) | (SEQ ID NO: 52) CGSYTTGAKSHVF |
| | | SLGSKY | GDS | |
| CD38 | VH | (SEQ ID NO: 53) | (SEQ ID NO: 54) | (SEQ ID NO: 55) CAKDKILWFGEPVFDYW |
| | | GFTFNSFA | ISGSGGGT | |
| | VL | (SEQ ID NO: 56) | (SEQ ID NO: 57) | (SEQ ID NO: 58) CQQRSNWPPTF |
| | | QSVSSY | DAS | |
| CD19 | VH | (SEQ ID NO: 59) | (SEQ ID NO: 60) | (SEQ ID NO: 61) CARGTYYYGTRVFDYW |
| | | GYTFTSYV | INPYNDGT | |
| | VL | (SEQ ID NO: 62) | (SEQ ID NO: 63) | (SEQ ID NO: 64) CMQHLEYPITF |
| | | KSLQNVNGNTY | RMS | |
| CD79b | VH | (SEQ ID NO: 65) | (SEQ ID NO: 66) | (SEQ ID NO: 67) CARAMGYW |
| | | GYTFTSYW | IDPSDSET | |
| | VL | (SEQ ID NO: 68) | (SEQ ID NO: 69) | (SEQ ID NO: 70) CWQGTHFPLTF |
| | | QSLLDSDGKTY | LVS | |
| CD40 | VH | (SEQ ID NO: 71) | (SEQ ID NO: 72) | (SEQ ID NO: 73) CVRTDGDYW |
| | | GFSLSRYS | MWGGGST | |
| | VL | (SEQ ID NO: 74) | (SEQ ID NO: 75) | (SEQ ID NO: 76) CSQSTHVPWTF |
| | | QSLVNSNGNTY | KVS | |
| CD80 | VH | (SEQ ID NO: 77) | (SEQ ID NO: 78) | (SEQ ID NO: 79) |
| | | GGSISGGYG | FYSSSGNT | CVRDRLFSVVGMVYNNWFDVW |
| | VL | (SEQ ID NO: 80) | (SEQ ID NO: 81) | (SEQ ID NO: 82) CQSYDSSLNAQVF |
| | | TSNIGGYD | DIN | |
| CD32b | VH | (SEQ ID NO: 83) | (SEQ ID NO: 84) | (SEQ ID NO: 85) CGALGLDYW |
| | | GFTFSDAW | IRNKAKNHAT | |
| | VL | (SEQ ID NO: 86) | (SEQ ID NO: 87) | (SEQ ID NO: 88) CLQYFSYPLTF |
| | | QEISGY | AAS | |

Reference
PDB ID: 5GGQ. Lee JY, Lee HT, Shin W, Chae J, Choi J, Kim SH, Lim H, Won Heo T, Park KY, Lee YJ, Ryu SE, Son JY, Lee JU, Heo YS. Structural basis of checkpoint blockade by monoclonal antibodies in cancer immunotherapy. Nat Commun. 2016 Oct 31;7: 13354. doi: 10.1038/ncomms13354. PMID: 27796306; PMCID: PMC5095608.
PDB ID: 5X8M. Lee HT, Lee JY, Lim H, Lee SH, Moon YJ, Pyo HJ, Ryu SE, Shin W, Heo YS. Molecular mechanism of PD-1/PD-L1 blockade via anti-PD-L1 antibodies atezolizumab and durvalumab. Sci Rep. 2017 Jul 17; 7(1): 5532. doi: 10.1038/s41598-017-06002-8. PMID: 28717238; PMCID: PMC5514103.
US2016/0376365A1
PDB ID: 5VKK. Ereno-Orbea J, Sicard T, Cui H, Mazhab-Jafari MT, Benlekbir S, Guarne A, Rubinstein JL, Julien
JP. Molecular basis of human CD22 function and therapeutic targeting. Nat Commun. 2017 Oct 2;8(1): 764. doi:
10.1038/s41467-017-00836-6. PMID: 28970495; PMCID: PMC5624926.
EP2843051A1 / US20150118237A1
PD ID: 7DUN. Yu, X.J., Wang, L., Yu, C.F. Crystal structure of Fab fragment of Daratumumab, unpublished
US 8,524,867 B2
WO 2017/214096 AI
US5,874,082
US 2002/0187146A1
WO 2017/214096 AI

Fresh PBMCs were isolated from heparin blood of healthy individuals by gradient separation. Cells were stained with monoclonal antibodies against the respective cell surface proteins and measured by flow cytometry and mass cytometry. B cells were defined as being CD19+. The markers CD27 and IgD were used to define naïve (IgD+CD27−), unswitched (IgD+CD27+), and switched memory (IgD− CD27+), as well as double negative B cells (IgD− CD27−). Plasmablast and plasma cells were defined as CD20^{low} CD27+CD38+, in addition plasma cells are CD138+. Mean fluorescence intensities (MFI) were used to define expression levels of the depicted markers in each subset.

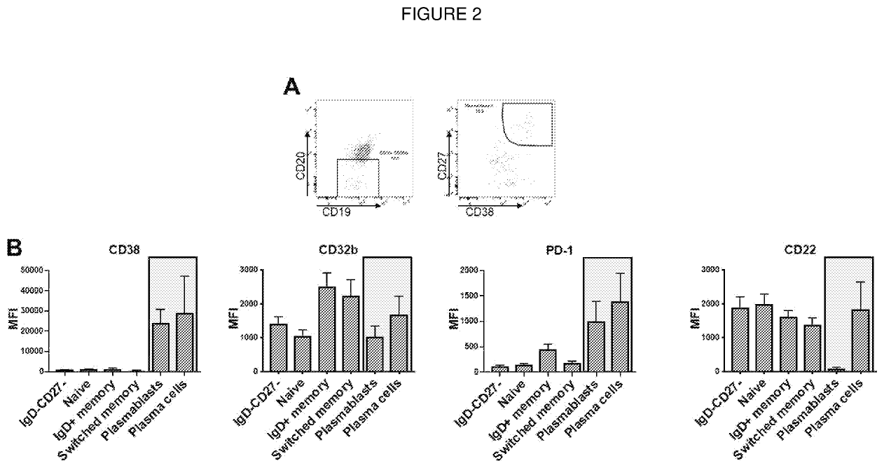

As shown in FIG. 2, screening data: (A) flow cytometry gating scheme. CD19+B cells are gated on CD20^{low} B cells and CD27^{high} as well as CD38^{high} B cells, encompassing activated plasmablasts and plasma cells. (B) Expression data of selected inhibitory cell surface receptors, including CD38, CD32b, PD-1, and CD22. Expression levels (mean fluorescence intensity, MFI) are shown for IgD CD27 double negative B cells, naïve B cells, unswitched and switched memory B cells as well as plasmablasts and plasma cells.

FIG. 3 provides a list of tested combinations of targeting molecules (activation markers) and inhibitory receptors; list of bi-specific formats used for bi-specific screening. Until otherwise specified, the IgG(H)-scFV (Morrison) was used in experiments. Expression and purification of bi-specific antibodies was done by WuXi Biologics, Wuxi, China. Antibodies were delivered in HBSS buffer, stored at −20° C. until use or 4° C. for up to one month.

Survival of B cells incubated with different bi-specific antibody molecules and with monospecific anti-CD38 antibody was tested, shown in FIG. 4(A) overall survival of B cells, and (B) survival of plasmablasts. Freshly isolated PBMC were cultured in the presence of R848 at 5 μg/ml as well as with the indicated mono- and bi-specific antibody compounds (at 10 μg/ml) for 48 h. Cells were stained with antibodies against B cell markers (CD19, CD20, CD27, CD38, IgD) and the live/dead dye Sytox green (Thermo Scientific). Sytox-negative cells (live cells) are shown as percentage of the indicated cell population.

Cell surface expression of activation markers on B cells after different stimulations with several bi-specific molecules and anti-CD38 tested as shown in FIG. 5(A) Cell surface expression of the early activation marker CD69, (B) cell surface expression of the late activation marker and antigen-presenting molecule HLA-DR. Freshly isolated PBMC were cultured in the presence of R848 at 5 μg/ml as well as with the indicated mono- and bi-specific antibody compounds at 10 μg/ml for 72 h. Cells were stained with antibodies against B cell markers (CD19, CD20, CD27, CD38, IgD) and as well as the indicated activation markers. Mean fluorescent intensities (MFI) are shown for the indicated markers for the whole CD19+B cell population. A bi-specific antibody with an inhibitory anti-PD1 specificity has been used to generate the evidence shown in FIGS. 4 and 5. It is likely, that efficacy of plasmablast inhibition will be increased by using a non-blocking anti-PD1 binding molecule.

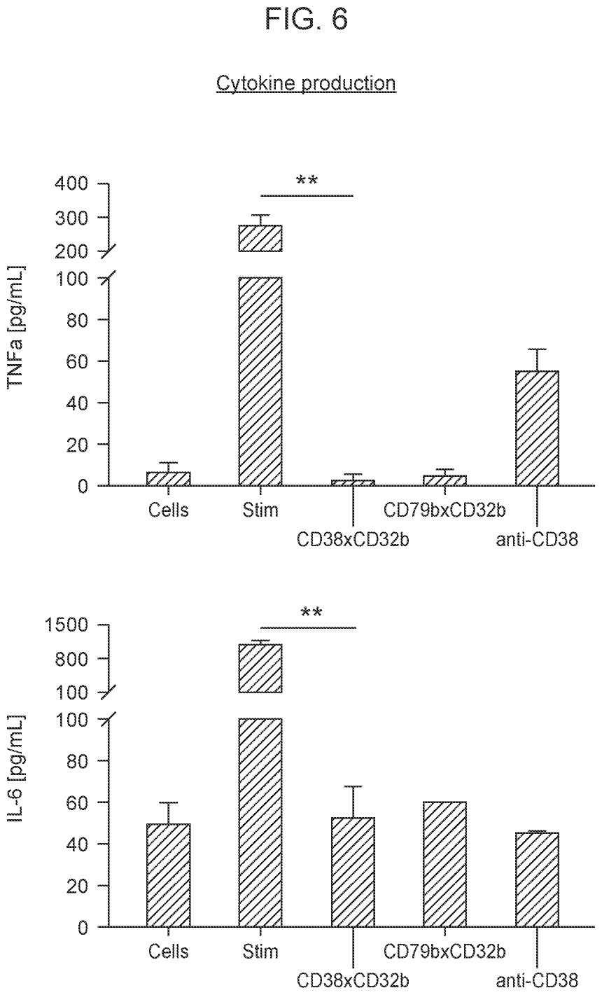

B cell secretion of the inflammatory cytokines TNF-alpha and Interleukin-6 (IL-6) upon incubation with bi-specific molecules CD38×CD32b and CD79×CD32b as well as mono-specific anti-CD38 is shown in FIG. 6. Freshly isolated PBMC were cultured in the presence of R848 at 5 μg/ml as well as with the indicated mono- and bi-specific antibody compounds at 10 μg/ml for 72 h. Cells were centrifuged for 5 min at 300 g, and supernatants were obtained and cytokine levels in supernatants were measured by sandwich ELISA (BD biosciences) and read with a Tecan microplate reader. Measured OD values were transformed to pg/ml according to a standard curve measured on the same plate. Values shown are means+/−standard deviations of triplicate measurements.

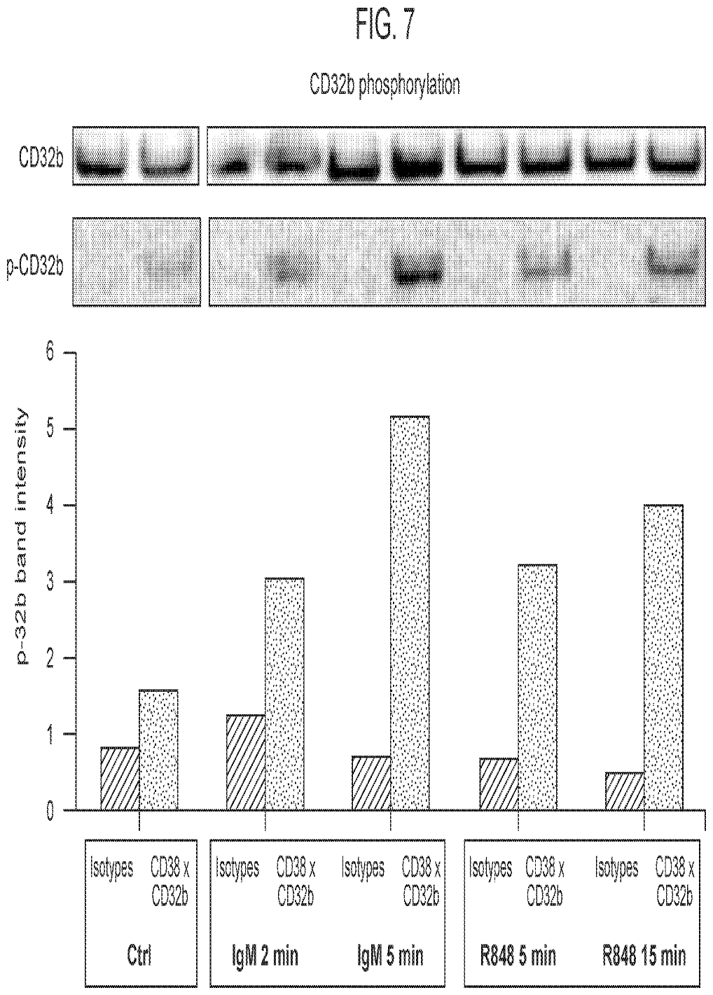

Activation (ITIM phosphorylation) of CD32b by the bi-specific antibody CD38×CD32b vs. isotype is shown in FIG. 7. Western blots for CD32b and phospho-CD32b are shown from B cell lysate upon stimulation with the bi-specific compound as well as the additional inflammatory stimulus. B cells were separated from PBMC by magnetic bead separation (negative selection, Stem Cell Technologies), seeded at 3×10⁶ cells/well in 96-well plates and stimulated 1 h after seeding with the bi-specific compound or isotype control (both 1 μg/ml) for an additional 1 h. B cells were then stimulated for the indicated time points (2-15 min) with anti-IgM (10 μg/ml) or R848 (5 μg/ml), centrifuged at 300 g for 5 min at 4° C., lysed in RIPA buffer with proteinase/phosphatase inhibitors, mixed with Laemmli buffer/10% beta-mercaptoethanol, run on a SDS-PAGE western blot gel, and transferred to a nitroglycerine membrane. Cells were sequentially stained, first for anti-phospho-CD32b and then for anti-CD32b (both Abcam) and developed with anti-Rabbit IgG HRP (Jackson ImmunoResearch) and ECL substrate (Thermo Scientific). Band intensities were evaluated with ImageJ.

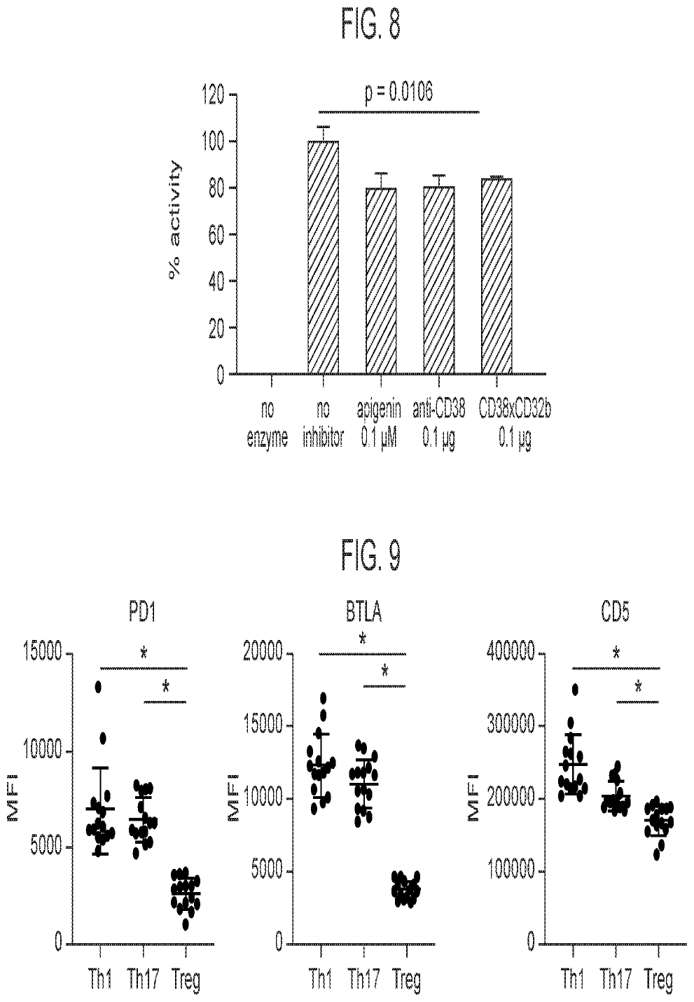

The CD38×CD32b bi-specific antibody inhibits CD38 enzymatic activity. Apigenin=small molecule inhibitor of CD38 NADase. Inhibition of enzymatic hydrolase activity was measured using a CD38 inhibitor screening assay (BPS bioscience), according to manufacturer instructions. In brief, 10 ng of recombinant CD38 is incubated in the appropriate buffer with antibody protein (0.1 μg) or the hydrolase inhibitor apigenin (0.1 μM, positive control) for 30 min., then &-NAD is added to each well for 12 min and measured in a fluorimeter with excitation at 300 nm and emission at 410 nm, data shown in FIG. 9.

Expression Levels of Suppressive Receptors in T cell Subsets is disclosed in Murphy K A, Bhamidipati K, Rubin S J S, Kipp L, Robinson W H, Lanz T V. “Immunomodulatory Receptors Are Differentially Expressed in B and T Cell Subsets Relevant to Autoimmune Disease.” Clinical Immunology 209 (Oct. 25, 2019): 108276. doi:10.1016/j.clim.2019.108276.) In brief, PBMC were stained for CD3, CD4, and CD8 as well as T cell subset defining cell surface markers: Th1 cells: CXCR3+, CCR4, Th17 cells: CCR6+, CD161+, Treg cells: CD25+, CD127−. Concomitantly, the indicated immunosuppressive cell surface receptors were stained and measured with flow cytometry. Mean fluorescence intensity is shown for each indicated cell surface receptor in each subset.

A list of all targeting molecules and inhibitory receptors, considered after the screen and tested in a bi-specific and/or multi-specific format using variable region sequences from Table 2. +/−indicates efficacy in the indicated suppressive assay. Proliferation, inflammatory function, and inhibition of B cell activation were assessed as described above and indicated in a scoring system from − to +++, shown in Table 3.

TABLE 3

| Activated [symbol] target | Inhibitory target | [symbol] | [symbol] | [symbol] | [symbol] |
|---|---|---|---|---|---|
| [symbol] | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| [symbol] | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| [symbol] | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| [symbol] | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| [symbol] | [symbol] | [symbol] | [symbol] | [symbol] | [symbol] |
| | [symbol] | [symbol] | [symbol] | [symbol] | [symbol] |

[symbol]

indicates data missing or illegible when filed

### Example 2

### A Study of the Effect of a Bi-Specific Inhibitor Antibody in Patients with Systemic Lupus Erythematosus

The purpose of this study is to determine the ability of a Bi-specific Inhibitor Antibody to maintain Systemic Lupus Erythematosus (SLE) disease activity improvement achieved by a brief course of disease-suppressing steroid therapy. The bi-specific inhibitor antibody, including a specificity set forth in Table 3, is administered by IV infusion for a total of up to 16 infusions, and compared to a placebo.

Primary outcome measurements are percentage of patients without loss of Systemic Lupus Erythematosus Disease Activity improvement on Day 225; and landmark proportion of patients without loss of systemic lupus erythematosus disease activity improvement on Day 225.

Secondary outcome measurements are percentage of patients and landmark proportion of patients without loss of Systemic lupus erythematosus disease activity improvement on day 169. Loss of improvement is defined as worsening of disease activity that in the opinion of the principal investigator requires a change in treatment (exclusive of a decrease in oral steroids) AND one of: SELENA-SLEDAI increase of >=4 points from maximal improvement OR Worsening of at least 1 BILAG A or B score OR New BILAG A or B score.

Inclusion criteria are adult patients with a diagnosis of SLE as defined by the ACR criteria; and history of a (+) ANA, (+) ENA or a (+) anti-dsDNA serology documented within one year prior to randomization.

### Example 3

### Study to Evaluate the Effect of a Bi-Specific Inhibitor Antibody on Disease Activity in Patients with IgG4-Related Disease (RD)

The purpose of this study is to determine the ability of a bi-specific Inhibitor Antibody to treat IgG4-related disease. The bi-specific inhibitor antibody, including a specificity set forth in Table 3, is administered by IV infusion for a total of up to 12 infusions, and compared to a placebo.

Primary outcome measurements are proportion of patient with an improvement in IgG4-RD activity. Improvement of disease activity as defined by a decrease of IgG4-RD responder index>=2 points from Day 1 pre-dose disease activity score. The IgG4-RD Responder Index Total Activity Score ranges from 0 to a maximum of 162. Higher scores represent greater (i.e. worse) disease activity. A score of 0 represents no disease activity other than residual fibrosis.

Secondary outcome measurements include number of patients experiencing a treatment-emergent adverse event as assessed by CTCAE v4.3 [Time Frame: Baseline Day 1 to Day 197]. The number of patients experiencing a treatment-emergent adverse event as assessed by CTCAE v4.3 will be tabulated according to MedDRA system-organ class and preferred term, intensity and causality.

Inclusion criteria are active IgG4-RD, compatible pattern of organ involvement consistent with IgG4-RD that cannot be attributed to other causes, histopathologically-proven diagnosis of IgG4-RD, peripheral blood plasmablast count >900 cells/mL and/or elevated IgG4-RD levels during screening.

### Example 4

### A Study to Evaluate Safety, Tolerability, and Efficacy of a Bi-Specific Inhibitor Antibody in Participants with Generalized Myasthenia Gravis

The purpose of this study is to determine the ability of a bi-specific Inhibitor Antibody to treat generalized myasthenia gravis. The bi-specific inhibitor antibody, including a specificity set forth in Table 3, is administered by IV or subcutaneous infusion once weekly for a total of up to 8 infusions, and compared to a placebo. Myasthenia gravis (MG) is an autoimmune disorder in which autoantibodies, such as those targeting the nicotinic acetylcholine receptor (AChR) or muscle specific kinase (MuSK), interfere with neuromuscular transmission, resulting in fatigue and weakness.

Primary outcome measurements are percentage of Participants with treatment emergent adverse events (TEAEs) and serious adverse events (SAEs), Grade 3 or higher TEAEs, AEs leading to discontinuation.

An Adverse Event (AE) is defined as any untoward medical occurrence in a clinical investigation participant administered a drug; it does not necessarily have to have a causal relationship with this treatment. An AE can therefore be any unfavorable and unintended sign (example, a clinically significant abnormal laboratory finding), symptom, or disease temporally associated with the use of a drug, whether or not it is considered related to the drug. A TEAE is defined as an AE with an onset that occurs after receiving study drug will be graded evaluated as per national cancer institute common terminology criteria for adverse events (NCI CTCAE), version 4.03.

Secondary outcomes include change from baseline in myasthenia gravis (MG) activities of daily living (MG-ADL) scale score [Time Frame: Baseline and Week 16]. Patient-reported scale to assess MG symptoms to evaluate capacity to perform activities of daily living. Each question was graded on a 3-point scale from 0=normal to 3=severe with a score of 0 to 24; the higher score indicates greater functional impairment and disability.

Secondary outcomes include change from baseline in quantitative Myasthenia Gravis (QMG) Scale Score [Time Frame: Baseline and Week 16]. Physician-reported scale to assess MG disease severity by quantifying several body functions by physical exam. Each question was graded on a 3-point scale from 0=normal to 3=severe with a score of 0 to 39; the higher score indicates greater disease burden.

Secondary outcomes include change from baseline in Anti-acetylcholine receptor (AChR) or Anti-Muscle-specific Tyrosine Kinase (MuSK) Antibody Levels [Time Frame: Baseline and Week 16]. Clinical laboratory evaluations (anti-AChR and anti-MuSK antibodies) will be tested to monitor disease activity.

Inclusion Criteria include diagnosis of Myasthenia Gravis (MG) supported by a positive serologic test for acetylcholine receptor (anti-AChR) or muscle-specific tyrosine kinase (anti-MuSK) antibodies at screening. Myasthenia Gravis Foundation of America (MGFA) clinical classification II to IV at screening. Myasthenia Gravis Activities of Daily Living (MG-ADL) total score of 6 or greater at screening, with at least 4 points attributed to nonocular items.

If receiving immunosuppressive drugs (ie, azathioprine, mycophenolate mofetil, methotrexate, cyclosporine, tacrolimus, cyclophosphamide), therapy must be ongoing for at least 6 months, with a stable dose at least 6 months before screening. If receiving oral corticosteroids, therapy must be ongoing for at least 3 months, with a stable dose at least 1 month before screening. Corticosteroids, including dexamethasone, must be given as oral, daily therapy as opposed to pulse therapy. If receiving cholinesterase inhibitors, therapy with a stable dose is required at least 2 weeks before screening. The doses of concomitant standard background therapy must be expected to remain stable throughout the study unless dose reduction is required due to toxicities. Allowed background therapy is defined as no more than a cholinesterase inhibitor±corticosteroid±one steroid-sparing immunosuppressive drug (limited to azathioprine, mycophenolate mofetil, methotrexate, cyclosporine, tacrolimus, or cyclophosphamide). Participants must be on at least one allowed background medication.

### Example 5

### A Study to Evaluate the Safety, Pharmacokinetics (PK), and Pharmacodynamics (PD) of a Bi-Specific Inhibitor Antibody in Combination with Standard Background Therapy in Participants with Moderate to Severe Systemic Lupus Erythematosus (SLE)

The purpose of this study is to evaluate the safety and tolerability of bi-specific inhibitor antibody, including a specificity set forth in Table 3, in comparison with matching placebo, administered once every 3 weeks over a 12-week dosing period in participants with active SLE who are receiving stable background therapy for SLE. This study will evaluate the safety and biologic activity of bi-specific inhibitor antibody, including a specificity set forth in Table 3, or matching placebo in combination with stable SLE background therapy.

The study will enroll approximately 24 participants across 3 sequentially enrolling cohorts. Each cohort will enroll 8 participants, where 6 participants will be assigned to drug injection, and 2 participants will be assigned to Placebo. Participants will receive drug or matching placebo in combination with principal investigator directed background therapy for SLE.

Participants will make multiple visits to the clinic, and will be followed up for the safety assessment for the additional 12 weeks up to Week 24 after receiving their last dose of study drug. Based on the clinical assessments, participants may complete or may advance to long-term safety follow up period for an additional 12-week safety monitoring period up to Week 36.

Primary outcome measurements include percentage of Participants with treatment emergent adverse events (TEAEs) and serious adverse events (SAEs), Grade 3 or higher TEAEs, AEs leading to discontinuation.

Secondary outcome measurements include change from baseline in immune cell subsets, including plasma cells, plasma blast (PBs), natural killer (NK) cells, B cells, T cells, monocytes, and total lymphocytes. CD38 expression and receptor occupancy will be evaluated for plasma cells, PBs, NK cells, B cells, T cells, and monocytes. Cytokine measurements will be assessed from baseline at different timepoints for placebo and each drug dose level.

Inclusion criteria are that the participant been diagnosed with SLE as defined by either the 2012 Systemic Lupus International Collaborating Clinics or the American College of Rheumatology diagnostic criteria. The participant has a systemic Lupus Erythematosus Disease Activity Index 2000 (SLEDAI-2K) score greater than or equal to (>=) 6. The participant is positive for anti-double-stranded deoxyribonucleic acid (dsDNA) antibodies and/or anti-extractable nuclear antigens (ENA) antibodies.

### Example 6

### A Study of Bi-Specific Inhibitor Antibody in Comparison with Interferon Beta-1a (Rebif) in Participants with Relapsing Multiple Sclerosis

This randomized, double-blind, double-dummy, parallel-group study will evaluate the efficacy and safety of bi-specific Inhibitor Antibody in comparison with interferon beta-1a (Rebif) in participants with relapsing multiple sclerosis. Participants will be randomized to receive either bi-specific Inhibitor Antibody 600 mg or matching placebo intravenous (IV) as 300 mg infusions on Days 1 and 15 for the first dose and as a single infusion of 600 mg for all subsequent infusions every 24 weeks, with placebo injections matching interferon beta-1a SC three times per week; or interferon beta-1a 44 mcg SC injections three times per week (with placebo infusions matching bi-specific Inhibitor Antibody infusions every 24 weeks). Planned duration of double-blind treatment is 96 weeks. Participants who complete the 96-week double-blind treatment will have an option to enter a single-group, active-treatment, open-label extension period, providing they fulfill the eligibility criteria.

Primary outcome measurements include annualized relapse rate in participants with relapsing multiple sclerosis at 96 weeks. ARR is protocol-defined and calculated as the total number of relapses for all participants in the treatment group divided by the total participant-years of exposure to that treatment.

Secondary outcome measurements include time to onset of confirmed disability progression for at least 12 weeks during the double blind treatment period. Disability progression is defined as an increase in the Expanded Disability Status Scale (EDSS) score of: A)>=1.0 point from the baseline EDSS score when the baseline score was less than or equal to (<=) 5.5 B)>=0.5 point from the baseline EDSS score when the baseline score was >5.5 The EDSS scale ranges from 0 (normal neurological exam) to 10 (death due to multiple sclerosis). This outcome measure was considered confirmatory only when results of both studies WA21092 and WA21093 were combined. Disability progression was considered confirmed when the increase in the EDSS was confirmed at a regularly scheduled visit at least 12 weeks after the initial documentation of neurological worsening. EDSS assessment and who were on treatment at time of clinical cut-off date were censored at the date of their last EDSS assessment.

Number of T1 Gadolinium (Gd)-Enhancing Lesions as Detected by Brain Magnetic Resonance Imaging (MRI) During the Double-Blind Treatment [Time Frame: Baseline up to Week 96]. The total number of T1 gadolinium-enhancing lesions for all participants in the treatment group was calculated as the sum of the individual number of lesions at Weeks 24, 48, and 96.

Number of New, and/or Enlarging T2 Hyperintense Lesions as Detected by Brain Magnetic Resonance Imaging (MRI) During the Double Blind Treatment [Time Frame: Baseline up to Week 96]. The total number of new and/or enlarging T2 lesions for all participants in the treatment group is calculated as the sum of the individual number of lesions at Weeks 24, 48, and 96.

Percentage of Participants With Confirmed Disability Improvement (CDI) for at Least 12 Weeks [Time Frame: Week 96]. Disability improvement is assessed only for the subgroup of participants with a baseline EDSS score of >=2.0. It was defined as a reduction in EDSS score of: A)>=1.0 from the baseline EDSS score when the baseline score was >=2 and <=5.5 B)>=0.5 when the baseline EDSS score >5.5. The EDSS scale ranges from 0 (normal neurological exam) to 10 (death due to multiple sclerosis). This outcome measure was considered confirmatory.

Inclusion criteria are diagnosis of multiple sclerosis, in accordance with the revised McDonald criteria (2010). At least 2 documented clinical attacks within the last 2 years prior to screening or one clinical attack in the years prior to screening (but not within 30 days prior to screening). Neurologic stability for greater than or equal to (>=) 30 days prior to both screening and baseline. Expanded Disability Status Scale (EDSS) score 0 to 5.5 inclusive

The examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A multi-specific antibody comprising at least two different antigen binding regions (ABR), a first ABR specifically that binds to an extracellular domain of an inhibitory receptor expressed on activated immune cells; and a second ABR that specifically binds to a targeting marker selected from a member of the T cell receptor (TCR) or B cell receptor (BCR) complex, a molecule associated with a member of the T cell receptor (TCR) or B cell receptor (BCR) complex, an activating co-receptor expressed on lymphocytes, or a subset-specific receptor.

2. The multi-specific antibody of claim 1, wherein the inhibitor receptor is selected from CD22, CD32b, CD95, BTLA, CD72, LAIR1, CD85j, LAG-3, PD1, TIGIT, CTLA4, TIM3, VISTA, CD38, 2B4, CD5, 4-1BB, CD2, CD49b, ICOS, TIM1, OX40, CD357, and CD30.

3. The multi-specific antibody of claim 1, wherein the targeting marker is selected from CD3, CD4, CD8, CD79b, CD19, CD20, CD38, CD138, CD95, CD93, CD69, CD30, PD-1, CD40, BCMA, GPRC5D, BTLA, LAG-3, CD70, PLD4, CD27, CD80, CD86, CD226, 4-1BB, CD2, CD49b, ICOS, TIM1, OX40, CD357, and CD30.

4. The multi-specific antibody of claim 1, wherein the antibody is comprised of at least one antibody variable domain from Table 2.

5. The multi-specific antibody of claim 1, wherein the antibody is a bi-specific antibody.

6. The bi-specific antibody of claim 5, wherein the antibodies are bivalent with a low amount of artificial mutations and low potential for immunogenicity.

7. The multi-specific antibody of claim 1, wherein the first ABR selectively binds to an inhibitory receptor selected from CD22, CD32b, CD95, BTLA, CD72, LAIR1, CD85j and LAG-3.

8. The multi-specific antibody of claim 7, wherein the antibody specifically binds to the inhibitory receptor CD32b.

9. The multi-specific antibody of claim 7, wherein the targeting marker is selected from CD38, CD138, CD30, CD95, CD93, BCMA, GPRC5D, PD-1, BTLA, LAG-3, CD70 and PLD4.

10. The multi-specific antibody of claim 1, wherein the targeting marker is CD38.

11. The multi-specific antibody of claim 10, wherein the antibody selectively binds to CD38 and CD22; to PD1 and to CD38; or to CD79b and to CD32b.

12-13. (canceled)

14. The multi-specific antibody of claim 1, wherein the first ABR selectively binds to an inhibitory receptor selected from PD-1, BTLA, CD5, and TIGIT.

15. The multi-specific antibody of claim 1, wherein the targeting marker is selected from CD3, CD4, CD8, CD38, CD69, CD30 and PD-1.

16. The multi-specific antibody of claim 1, wherein the antibody binds to the combination of CD19 and CD22; CD19 and CD32b; CD19 and PD-1; CD20 and CD32b; CD79b and CD22; CD79b and CD32b; CD79b and CD30; CD79b and PD-1; CD69 and CD22; CD69 and PD-1; CD69 and CD32b; CD69 and PD-1; CD69 and TIGIT; CD69 and PD-L1; PD-1 and CD32b; PD-1 and CD30; CD-1 and CD80; CD40 and CD22; or CD40 and PD-1.

17. A pharmaceutical composition comprising a multi-specific antibody according to claim 1.

18. A method for treating inflammatory disease, comprising administering an effective dose of a pharmaceutical composition of claim 16.

19. The method of claim 17, wherein the inflammatory disease is an autoimmune disease.

20. The method of claim 18, wherein the disease is associated with adverse activated B cells responses.

21. The method of claim 19, wherein the disease is rheumatoid arthritis; myasthenia gravis; systemic lupus erythematosus (SLE), ankylosing spondylitis (AS); psoriatic arthritis (PsA); scleroderma; Sjogren's syndrome, or IgG4-related disease.
